SEQUENCE LISTING (172 sequences)

SEQ ID NO: 1 - DNA, 58 bp, synthetic construct
  source 1..58: mol_type = other DNA
gagaggtatgtggcctttgaaactacctccctgaatttacatcatcatacatcatcat

SEQ ID NO: 2 - DNA, 53 bp, synthetic construct
  source 1..53: mol_type = other DNA
aagcaaaggaggatggggagactccgccggtttacatcatcatacatcatcat

SEQ ID NO: 3 - DNA, 60 bp, synthetic construct
  source 1..60: mol_type = other DNA
gcagctggaagagagatggttttgagtttcatttggctttacatcatcatacatcatcat

SEQ ID NO: 4 - DNA, 53 bp, synthetic construct
  source 1..53: mol_type = other DNA
gatagggtcgggcacggagcacaggcagagtttacatcatcatacatcatcat

SEQ ID NO: 5 - DNA, 60 bp, synthetic construct
  source 1..60: mol_type = other DNA
ggcttgttgcatctcacctacaaagcttcaagtttcttttacatcatcatacatcatcat

SEQ ID NO: 6 - DNA, 60 bp, synthetic construct
  source 1..60: mol_type = other DNA
tggccaatgaggtttctaaaaggcccatccaaagatatttacatcatcatacatcatcat

SEQ ID NO: 7 - DNA, 59 bp, synthetic construct
  source 1..59: mol_type = other DNA
ctggaagttttctgagttcctgccccaagattcaagtttacatcatcatacatcatcat

SEQ ID NO: 8 - DNA, 53 bp, synthetic construct
  source 1..53: mol_type = other DNA
ctagctcctgagtcttgcaccaagcgcgcgtttacatcatcatacatcatcat

SEQ ID NO: 9 - DNA, 53 bp, synthetic construct
  source 1..53: mol_type = other DNA
cagagtagtgggtggcaccgcgcctcctagtttacatcatcatacatcatcat

SEQ ID NO: 10 - DNA, 53 bp, synthetic construct
  source 1..53: mol_type = other DNA
tgggtcttcccctgggtctcttaggagaggtttacatcatcatacatcatcat

SEQ ID NO: 11 - DNA, 60 bp, synthetic construct
  source 1..60: mol_type = other DNA
ggaacagggagaaagtcaactgcagttaaacgtcttgtttacatcatcatacatcatcat

SEQ ID NO: 12 - DNA, 60 bp, synthetic construct
  source 1..60: mol_type = other DNA
tccttttgaagtctggaggtaggtctttgcaagtcaatttacatcatcatacatcatcat

SEQ ID NO: 13 - DNA, 53 bp, synthetic construct
  source 1..53: mol_type = other DNA
atcaggagttgcgggcaccgggagcacttctttacatcatcatacatcatcat

SEQ ID NO: 14 - DNA, 59 bp, synthetic construct
  source 1..59: mol_type = other DNA
cgtcatgagaaactacctcctggcccagttgataattttacatcatcatacatcatcat

SEQ ID NO: 15 - DNA, 54 bp, synthetic construct
  source 1..54: mol_type = other DNA
ccatttcctaggtctgcctcttggccgtttttttacatcatcatacatcatcat

SEQ ID NO: 16 - DNA, 53 bp, synthetic construct
  source 1..53: mol_type = other DNA
aggttgtccctgggctgaagtggacggctgtttacatcatcatacatcatcat

SEQ ID NO: 17 - DNA, 56 bp, synthetic construct
  source 1..56: mol_type = other DNA
tgattcacaacagtgagcgatctgccagtctcatttacatcatcatacatcatcat

SEQ ID NO: 18 - DNA, 53 bp, synthetic construct
  source 1..53: mol_type = other DNA
ctttgaggtcgcagaggctcctctcgcgggtttacatcatcatacatcatcat

SEQ ID NO: 19 - DNA, 59 bp, synthetic construct
  source 1..59: mol_type = other DNA
ccatgtttggccaacatgagcaattgcttccatttttttacatcatcatacatcatcat

SEQ ID NO: 20 - DNA, 60 bp, synthetic construct
  source 1..60: mol_type = other DNA
gctgcgaccaacaacagcaaagaacagtcctataaaatttacatcatcatacatcatcat

SEQ ID NO: 21 - DNA, 60 bp, synthetic construct
  source 1..60: mol_type = other DNA
agggaagagacttggagtgaattgtgtcccttgaaattttacatcatcatacatcatcat

SEQ ID NO: 22 - DNA, 60 bp, synthetic construct
  source 1..60: mol_type = other DNA
tggggatcacagaggctgttatgtttattgtgcagaatttacatcatcatacatcatcat

SEQ ID NO: 23 - DNA, 53 bp, synthetic construct
  source 1..53: mol_type = other DNA
gagatgaggtgatagggctggggacgccggtttacatcatcatacatcatcat

SEQ ID NO: 24 - DNA, 53 bp, synthetic construct
  source 1..53: mol_type = other DNA
gctgaggtagaaatcgaacgttggcgcccctttacatcatcatacatcatcat

SEQ ID NO: 25 - DNA, 58 bp, synthetic construct
  source 1..58: mol_type = other DNA
tgcagtgtcttctcccttcaaaagatccaactgcttttacatcatcatacatcatcat

SEQ ID NO: 26 - DNA, 59 bp, synthetic construct
  source 1..59: mol_type = other DNA
acctgggactgtgaagggattacaaagaaggtgatttttacatcatcatacatcatcat

SEQ ID NO: 27 - DNA, 60 bp, synthetic construct
  source 1..60: mol_type = other DNA
cattgttaagctgtgtcccctgtgtttacagcagttttttacatcatcatacatcatcat

SEQ ID NO: 28 - DNA, 60 bp, synthetic construct
  source 1..60: mol_type = other DNA
acccgcccactcttgcttatcagatcaacaaactatttttacatcatcatacatcatcat

SEQ ID NO: 29 - DNA, 59 bp, synthetic construct
  source 1..59: mol_type = other DNA
tgcagtgcagtgattgactaaacccaattcggtttttttacatcatcatacatcatcat

SEQ ID NO: 30 - DNA, 57 bp, synthetic construct
  source 1..57: mol_type = other DNA
cgacgtcacaagtgtgacacgtttcttgtttgcatttacatcatcatacatcatcat

SEQ ID NO: 31 - DNA, 60 bp, synthetic construct
  source 1..60: mol_type = other DNA
acagtgttacacaccgcgttcttcctatatgaatgcctttacatcatcatacatcatcat

SEQ ID NO: 32 - DNA, 55 bp, synthetic construct
  source 1..55: mol_type = other DNA
gggagtgggttggggtggtatttgaggtgtactttacatcatcatacatcatcat

SEQ ID NO: 33 - skipped

SEQ ID NO: 34 - DNA, 60 bp, synthetic construct
  source 1..60: mol_type = other DNA
gccgaagaaaagcaccttggtcaaccatcttatgtcatttacatcatcatacatcatcat

SEQ ID NO: 35 - DNA, 60 bp, synthetic construct
  source 1..60: mol_type = other DNA
agtggggatatattgcatctctggctaaaagtgcagttttacatcatcatacatcatcat

SEQ ID NO: 36 - DNA, 53 bp, synthetic construct
  source 1..53: mol_type = other DNA
gtgctctctcccgcaccagtcatcaccgggtttacatcatcatacatcatcat

SEQ ID NO: 37 - DNA, 55 bp, synthetic construct
  source 1..55: mol_type = other DNA
ccgaatacttgagctcgctgttctcgggattgtttacatcatcatacatcatcat

SEQ ID NO: 38 - DNA, 53 bp, synthetic construct
  source 1..53: mol_type = other DNA
aaagcagacacgagtggagtgaggcccgcgtttacatcatcatacatcatcat

SEQ ID NO: 39 - DNA, 53 bp, synthetic construct
  source 1..53: mol_type = other DNA
tctgtcagcgcagtcgccactgtctggacctttacatcatcatacatcatcat

SEQ ID NO: 40 - DNA, 56 bp, synthetic construct
  source 1..56: mol_type = other DNA
cgagactgacgcctatgtagagcccatcttgactttacatcatcatacatcatcat

SEQ ID NO: 41 - DNA, 53 bp, synthetic construct
  source 1..53: mol_type = other DNA
aatccggtcgcacggaggacgtgacttcggtttacatcatcatacatcatcat

SEQ ID NO: 42 - DNA, 53 bp, synthetic construct
  source 1..53: mol_type = other DNA
tccatagcaggcaaggcccaggtcctagtctttacatcatcatacatcatcat

SEQ ID NO: 43 - DNA, 59 bp, synthetic construct
  source 1..59: mol_type = other DNA
ccatgaacgcacatgaaatctggatgtggaaaaggttttacatcatcatacatcatcat

SEQ ID NO: 44 - DNA, 60 bp, synthetic construct
  source 1..60: mol_type = other DNA
tccgactgacttcagatcagccacacaatatgaactttttacatcatcatacatcatcat

SEQ ID NO: 45 - DNA, 60 bp, synthetic construct
  source 1..60: mol_type = other DNA
cctgcctgtgggttagtcacactatacagacttcatctttacatcatcatacatcatcat

SEQ ID NO: 46 - DNA, 60 bp, synthetic construct
  source 1..60: mol_type = other DNA
gccttaaagcattttgcaaacttcacgtgcacttgtttttacatcatcatacatcatcat

SEQ ID NO: 47 - DNA, 60 bp, synthetic construct
  source 1..60: mol_type = other DNA
acaaaacggctccaaaaggactatgctttgaaaggaatttacatcatcatacatcatcat

SEQ ID NO: 48 - DNA, 60 bp, synthetic construct
  source 1..60: mol_type = other DNA
gcacatggagagtagtaactaaccccatgtgtcaacttttacatcatcatacatcatcat

SEQ ID NO: 49 - DNA, 60 bp, synthetic construct
  source 1..60: mol_type = other DNA
gccctgttcgtgggccagaatatatttcattcacaaatttacatcatcatacatcatcat

SEQ ID NO: 50 - DNA, 59 bp, synthetic construct
  source 1..59: mol_type = other DNA
agctttgagacttcaaggcacgttgcaaaaggaaaatttacatcatcatacatcatcat

SEQ ID NO: 51 - DNA, 60 bp, synthetic construct
  source 1..60: mol_type = other DNA
ggccactggaatcatttctacctatcttctgttggggtttacatcatcatacatcatcat

SEQ ID NO: 52 - DNA, 59 bp, synthetic construct
  source 1..59: mol_type = other DNA
tcttctctcactctttcagcaagcctcttgggaaaatttacatcatcatacatcatcat

SEQ ID NO: 53 - DNA, 53 bp, synthetic construct
  source 1..53: mol_type = other DNA
ctggcttctggaagtggagcagggtccgcctttacatcatcatacatcatcat

SEQ ID NO: 54 - DNA, 53 bp, synthetic construct
  source 1..53: mol_type = other DNA
aaacaaacagcgctcgccgaagggtgccagtttacatcatcatacatcatcat

SEQ ID NO: 55 - DNA, 53 bp, synthetic construct
  source 1..53: mol_type = other DNA
ctgggctcctgattggactcaccgagcccttttacatcatcatacatcatcat

SEQ ID NO: 56 - DNA, 53 bp, synthetic construct
  source 1..53: mol_type = other DNA
cctcttactgctctgccggaaaactgcagctttacatcatcatacatcatcat

SEQ ID NO: 57 - DNA, 53 bp, synthetic construct
  source 1..53: mol_type = other DNA
gggtctgcgccgcgctgctggtgaataaaatttacatcatcatacatcatcat

SEQ ID NO: 58 - DNA, 53 bp, synthetic construct
  source 1..53: mol_type = other DNA
ctggaaaggctctggagtcgctggcgtccgtttacatcatcatacatcatcat

SEQ ID NO: 59 - DNA, 53 bp, synthetic construct
  source 1..53: mol_type = other DNA
aagcgtaggaactgagcagcagcgagcgggtttacatcatcatacatcatcat

SEQ ID NO: 60 - DNA, 53 bp, synthetic construct
  source 1..53: mol_type = other DNA
agcaagtcctcccagctcgacaggttgccctttacatcatcatacatcatcat

SEQ ID NO: 61 - DNA, 53 bp, synthetic construct
  source 1..53: mol_type = other DNA
gtcgcggcttggtcgaggtcagtgaacagctttacatcatcatacatcatcat

SEQ ID NO: 62 - DNA, 53 bp, synthetic construct
  source 1..53: mol_type = other DNA
tacatctcctccggctgctcgggtgcgaagtttacatcatcatacatcatcat

SEQ ID NO: 63 - DNA, 53 bp, synthetic construct
  source 1..53: mol_type = other DNA
gtctggatggagccgcagttcacgcactcgtttacatcatcatacatcatcat

SEQ ID NO: 64 - DNA, 53 bp, synthetic construct
  source 1..53: mol_type = other DNA
tagagcccgcaggcgttgcacaggtagtggtttacatcatcatacatcatcat

SEQ ID NO: 65 - DNA, 53 bp, synthetic construct
  source 1..53: mol_type = other DNA
atgaggggccggctgaggccgttcatcttgtttacatcatcatacatcatcat

SEQ ID NO: 66 - DNA, 54 bp, synthetic construct
  source 1..54: mol_type = other DNA
tggtgtgacagttggcacaggacaatccaagtttacatcatcatacatcatcat

SEQ ID NO: 67 - DNA, 53 bp, synthetic construct
  source 1..53: mol_type = other DNA
cggcgtttctgcgccataaggtggtagttgtttacatcatcatacatcatcat

SEQ ID NO: 68 - DNA, 53 bp, synthetic construct
  source 1..53: mol_type = other DNA
gtccacaagcattgcacacgggttcacccttttacatcatcatacatcatcat

SEQ ID NO: 69 - DNA, 60 bp, synthetic construct
  source 1..60: mol_type = other DNA
aggtggaagttggagtcatgggaatggaattattgcttttacatcatcatacatcatcat

SEQ ID NO: 70 - DNA, 55 bp, synthetic construct
  source 1..55: mol_type = other DNA
taaaagtgcagcgtgaatgcagagatggcagttttacatcatcatacatcatcat

SEQ ID NO: 71 - DNA, 53 bp, synthetic construct
  source 1..53: mol_type = other DNA
ccgaaccaagataatgcacctcggctagcctttacatcatcatacatcatcat

SEQ ID NO: 72 - DNA, 60 bp, synthetic construct
  source 1..60: mol_type = other DNA
ggtagtattgaggagtggggattattgcatctctggctttacatcatcatacatcatcat

SEQ ID NO: 73 - DNA, 60 bp, synthetic construct
  source 1..60: mol_type = other DNA
tttcttcaaccacacacccttgcttatcagatccacatttacatcatcatacatcatcat

SEQ ID NO: 74 - DNA, 60 bp, synthetic construct
  source 1..60: mol_type = other DNA
tactcagctagttatctccgatgcttcaaggctgacatttacatcatcatacatcatcat

SEQ ID NO: 75 - DNA, 60 bp, synthetic construct
  source 1..60: mol_type = other DNA
ttatggctaagtggcttttggctgtgtagatttcccgtttacatcatcatacatcatcat

SEQ ID NO: 76 - DNA, 53 bp, synthetic construct
  source 1..53: mol_type = other DNA
aggcgggaaagagagcttgtgggcttatcatttacatcatcatacatcatcat

SEQ ID NO: 77 - DNA, 60 bp, synthetic construct
  source 1..60: mol_type = other DNA
aacaagtgcagggctgtaaattcactgtagcattcagtttacatcatcatacatcatcat

SEQ ID NO: 78 - DNA, 60 bp, synthetic construct
  source 1..60: mol_type = other DNA
tggaggtagttccttgcaaatcaggaagtccatttgttttacatcatcatacatcatcat

SEQ ID NO: 79 - DNA, 58 bp, synthetic construct
  source 1..58: mol_type = other DNA
cgtggcagttaagaataactggtcactgcagtgtctttacatcatcatacatcatcat

SEQ ID NO: 80 - DNA, 60 bp, synthetic construct
  source 1..60: mol_type = other DNA
tatagcaaaagagatttcggtttcgctcctcccatgttttacatcatcatacatcatcat

SEQ ID NO: 81 - DNA, 59 bp, synthetic construct
  source 1..59: mol_type = other DNA
atccaactgatgctgaggtagaaattgaatgtcggcgtttacatcatcatacatcatca

SEQ ID NO: 82 - DNA, 59 bp, synthetic construct
  source 1..59: mol_type = other DNA
catcacttgggtgggttcattgggtactgttaaccttttacatcatcatacatcatcat

SEQ ID NO: 83 - DNA, 53 bp, synthetic construct
  source 1..53: mol_type = other DNA
cccctaatcctcctctgcggactcttgcgatttacatcatcatacatcatcat

SEQ ID NO: 84 - DNA, 60 bp, synthetic construct
  source 1..60: mol_type = other DNA
aagagtacagagaagaatggtcgagaactggggatggtttacatcatcatacatcatcat

SEQ ID NO: 85 - DNA, 60 bp, synthetic construct
  source 1..60: mol_type = other DNA
tcaaaatgaaaggtgttacaggatgtaacacgcaggctttacatcatcatacatcatcat

SEQ ID NO: 86 - DNA, 55 bp, synthetic construct
  source 1..55: mol_type = other DNA
ctgagatgccgacccagtaaggtttggtttggtttacatcatcatacatcatcat

SEQ ID NO: 87 - DNA, 58 bp, synthetic construct
  source 1..58: mol_type = other DNA
ttcatctgaggtaagtggtactgcggtctttcacgtttacatcatcatacatcatcat

SEQ ID NO: 88 - DNA, 58 bp, synthetic construct
  source 1..58: mol_type = other DNA
ttttcctttctcttgcagggcttgtgtctagcagctttacatcatcatacatcatcat

SEQ ID NO: 89 - DNA, 58 bp, synthetic construct
  source 1..58: mol_type = other DNA
ctcctgctgctgtgtatgagtccataaatggaggatttacatcatcatacatcatcat

SEQ ID NO: 90 - DNA, 60 bp, synthetic construct
  source 1..60: mol_type = other DNA
tggtgtcctaagcagattttctatgatcatggtggggtttacatcatcatacatcatcat

SEQ ID NO: 91 - DNA, 60 bp, synthetic construct
  source 1..60: mol_type = other DNA
aatgtaacaatctagcaggaaagctgccacaagcatttttacatcatcatacatcatcat

SEQ ID NO: 92 - DNA, 54 bp, synthetic construct
  source 1..54: mol_type = other DNA
gtggcatctgagtggacacagagtcacacactttacatcatcatacatcatcat

SEQ ID NO: 93 - DNA, 53 bp, synthetic construct
  source 1..53: mol_type = other DNA
cccggtcaccagggtgccggtttagggttttttacatcatcatacatcatcat

SEQ ID NO: 94 - DNA, 60 bp, synthetic construct
  source 1..60: mol_type = other DNA
ataacttcatcagagatggaaattgctcggtgtgccgtttacatcatcatacatcatcat

SEQ ID NO: 95 - DNA, 57 bp, synthetic construct
  source 1..57: mol_type = other DNA
gcatgtagtgactgattaagcccaccacagaggttttacatcatcatacatcatcat

SEQ ID NO: 96 - DNA, 53 bp, synthetic construct
  source 1..53: mol_type = other DNA
agtctggccaacacgagaagctgcttcagttttacatcatcatacatcatcat

SEQ ID NO: 97 - DNA, 54 bp, synthetic construct
  source 1..54: mol_type = other DNA
cggggcccacagaggctgttatgtttattgttttacatcatcatacatcatcat

SEQ ID NO: 98 - DNA, 60 bp, synthetic construct
  source 1..60: mol_type = other DNA
ttttatgagctggccaaaatgaggtttctgtaaggcctttacatcatcatacatcatcat

SEQ ID NO: 99 - DNA, 60 bp, synthetic construct
  source 1..60: mol_type = other DNA
ataatccactagagaaggtattgccacaagcaagccgtttacatcatcatacatcatcat

SEQ ID NO: 100 - DNA, 60 bp, synthetic construct
  source 1..60: mol_type = other DNA
tttaatctctccatagctcctttggcgactcacaacatttacatcatcatacatcatcat

SEQ ID NO: 101 - DNA, 53 bp, synthetic construct
  source 1..53: mol_type = other DNA
gtgagccatctgccagtctcagagcggcactttacatcatcatacatcatcat

SEQ ID NO: 102 - DNA, 56 bp, synthetic construct
  source 1..56: mol_type = other DNA
aacagagaacgaagtcagagggcacacaaaggatttacatcatcatacatcatcat

SEQ ID NO: 103 - DNA, 56 bp, synthetic construct
  source 1..56: mol_type = other DNA
gtggcctttgaagtaacctccctgaaaacagcttttacatcatcatacatcatcat

SEQ ID NO: 104 - DNA, 58 bp, synthetic construct
  source 1..58: mol_type = other DNA
acggggagaaagttggatgcagttaatcgtcttgatttacatcatcatacatcatcat

SEQ ID NO: 105 - DNA, 60 bp, synthetic construct
  source 1..60: mol_type = other DNA
tactgacacaagccactatgagaaactacctcctggctttacatcatcatacatcatcat

SEQ ID NO: 106 - DNA, 53 bp, synthetic construct
  source 1..53: mol_type = other DNA
ttttgctgcctttgggtcttcctctgggtctttacatcatcatacatcatcat

SEQ ID NO: 107 - DNA, 55 bp, synthetic construct
  source 1..55: mol_type = other DNA
ccagtgggtaacactctggaaagtcagcacactttacatcatcatacatcatcat

SEQ ID NO: 108 - DNA, 53 bp, synthetic construct
  source 1..53: mol_type = other DNA
caggttgtccctgggctgatgtggacggcttttacatcatcatacatcatcat

SEQ ID NO: 109 - DNA, 59 bp, synthetic construct
  source 1..59: mol_type = other DNA
acacaagaagcgtccaaaacaaatctgcttgagacatttacatcatcatacatcatcat

SEQ ID NO: 110 - DNA, 59 bp, synthetic construct
  source 1..59: mol_type = other DNA
ttttcattgcattaggcaaaaggctgagctggttgttttacatcatcatacatcatcat

SEQ ID NO: 111 - DNA, 53 bp, synthetic construct
  source 1..53: mol_type = other DNA
cgcccagcacctttcgggagacgggatgtatttacatcatcatacatcatcat

SEQ ID NO: 112 - DNA, 60 bp, synthetic construct
  source 1..60: mol_type = other DNA
aaatgtttcaatgagggagaggtgaggtttgagctcctttacatcatcatacatcatcat

SEQ ID NO: 113 - DNA, 53 bp, synthetic construct
  source 1..53: mol_type = other DNA
gagagctggagagggcaagagcggcgagagtttacatcatcatacatcatcat

SEQ ID NO: 114 - DNA, 60 bp, synthetic construct
  source 1..60: mol_type = other DNA
tgagtgatataggctgcaagttcgttcctttggaagttttacatcatcatacatcatcat

SEQ ID NO: 115 - DNA, 53 bp, synthetic construct
  source 1..53: mol_type = other DNA
gagtgcgccggggactactgctgctgcttatttacatcatcatacatcatcat

SEQ ID NO: 116 - DNA, 56 bp, synthetic construct
  source 1..56: mol_type = other DNA
taaacaaacagcgctagccgaagggtgccaggctttacatcatcatacatcatcat

SEQ ID NO: 117 - DNA, 55 bp, synthetic construct
  source 1..55: mol_type = other DNA
cggggtctgctagtcgctgctggtgaataaaatttacatcatcatacatcatcat

SEQ ID NO: 118 - DNA, 54 bp, synthetic construct
  source 1..54: mol_type = other DNA
gtccggcgctactccaacctgacttttgatttttacatcatcatacatcatcat

SEQ ID NO: 119 - DNA, 53 bp, synthetic construct
  source 1..53: mol_type = other DNA
cctgcgctcctgattggactcaccgagccctttacatcatcatacatcatcat

SEQ ID NO: 120 - DNA, 53 bp, synthetic construct
  source 1..53: mol_type = other DNA
gcctcttactgctctgccggaaaactgcagtttacatcatcatacatcatcat

SEQ ID NO: 121 - DNA, 60 bp, synthetic construct
  source 1..60: mol_type = other DNA
tcctgcaaaagcccatctcttcttacttcagtaagcttttacatcatcatacatcatcat

SEQ ID NO: 122 - DNA, 53 bp, synthetic construct
  source 1..53: mol_type = other DNA
gagtcagaggcaggaagcagcccaggctggtttacatcatcatacatcatcat

SEQ ID NO: 123 - DNA, 53 bp, synthetic construct
  source 1..53: mol_type = other DNA
tgtcacagtttctcccactggtatggggcatttacatcatcatacatcatcat

SEQ ID NO: 124 - DNA, 53 bp, synthetic construct
  source 1..53: mol_type = other DNA
atcttccttagcagacaaggccccggtcattttacatcatcatacatcatcat

SEQ ID NO: 125 - DNA, 60 bp, synthetic construct
  source 1..60: mol_type = other DNA
cgaggttgatcacaagaagcacatgatttggagcaattttacatcatcatacatcatcat

SEQ ID NO: 126 - DNA, 55 bp, synthetic construct
  source 1..55: mol_type = other DNA
ggtggtcggggatgaatgggttctgggataactttacatcatcatacatcatcat

SEQ ID NO: 127 - DNA, 54 bp, synthetic construct
  source 1..54: mol_type = other DNA
tcctctccacgaacgcttgtgaaatgtgcactttacatcatcatacatcatcat

SEQ ID NO: 128 - DNA, 54 bp, synthetic construct
  source 1..54: mol_type = other DNA
cctccaggatagaccaaatggctcccagtgctttacatcatcatacatcatcat

SEQ ID NO: 129 - DNA, 60 bp, synthetic construct
  source 1..60: mol_type = other DNA
gccacgttacgatgaacgttgagataagtcagtcagttttacatcatcatacatcatcat

SEQ ID NO: 130 - DNA, 60 bp, synthetic construct
  source 1..60: mol_type = other DNA
cacagaagtgggctgtgagtgtaagaagcatatgtcttttacatcatcatacatcatcat

SEQ ID NO: 131 - DNA, 60 bp, synthetic construct
  source 1..60: mol_type = other DNA
tcagtaaaagaacggggactgtgttggtgttcttgtatttacatcatcatacatcatcat

SEQ ID NO: 132 - DNA, 59 bp, synthetic construct
  source 1..59: mol_type = other DNA
cacgtggtacaggcgtcaagagtgttacagatactttttacatcatcatacatcatcat

SEQ ID NO: 133 - DNA, 53 bp, synthetic construct
  source 1..53: mol_type = other DNA
gagcaggaggaggacgaagacgagatggggtttacatcatcatacatcatcat

SEQ ID NO: 134 - DNA, 53 bp, synthetic construct
  source 1..53: mol_type = other DNA
gggatgcgaggcgtaggggctgagcaagagtttacatcatcatacatcatcat

SEQ ID NO: 135 - DNA, 53 bp, synthetic construct
  source 1..53: mol_type = other DNA
aacaggtcctcccaagtcgacagggcgctctttacatcatcatacatcatcat

SEQ ID NO: 136 - DNA, 53 bp, synthetic construct
  source 1..53: mol_type = other DNA
gtcgcggcctgatcgaggtcagtgaagagctttacatcatcatacatcatcat

SEQ ID NO: 137 - DNA, 53 bp, synthetic construct
  source 1..53: mol_type = other DNA
tacatttcctccggctgctcggccgcgaagtttacatcatcatacatcatcat

SEQ ID NO: 138 - DNA, 53 bp, synthetic construct
  source 1..53: mol_type = other DNA
tgaaggtagggcaggccggacagcatggagtttacatcatcatacatcatcat

SEQ ID NO: 139 - DNA, 53 bp, synthetic construct
  source 1..53: mol_type = other DNA
gagctgtactggtgctcccggccacccatgtttacatcatcatacatcatcat

SEQ ID NO: 140 - DNA, 54 bp, synthetic construct
  source 1..54: mol_type = other DNA
atggtggtggtggtacgttccgttcagcggctttacatcatcatacatcatcat

SEQ ID NO: 141 - DNA, 53 bp, synthetic construct
  source 1..53: mol_type = other DNA
catgtagggcgagtaggtcgggtgatggtgtttacatcatcatacatcatcat

SEQ ID NO: 142 - DNA, 53 bp, synthetic construct
  source 1..53: mol_type = other DNA
gaagggtcctgctggccaggcaggagtcagtttacatcatcatacatcatcat

SEQ ID NO: 143 - DNA, 53 bp, synthetic construct
  source 1..53: mol_type = other DNA
gccctgtaagctgtggagcaccggcgtttctttacatcatcatacatcatcat

SEQ ID NO: 144 - DNA, 53 bp, synthetic construct; identical to SEQ ID NO: 63
  source 1..53: mol_type = other DNA

SEQ ID NO: 145 - DNA, 53 bp, synthetic construct
  source 1..53: mol_type = other DNA
atgaccggtgccgtctcgtctccacagtggtttacatcatcatacatcatcat

SEQ ID NO: 146 - DNA, 53 bp, synthetic construct
  source 1..53: mol_type = other DNA
gctgtagagaccgcatgcattgcacaggtatttacatcatcatacatcatcat

SEQ ID NO: 147 - DNA, 53 bp, synthetic construct
  source 1..53: mol_type = other DNA
gatgaggggcctgctgaggccattcatctttttacatcatcatacatcatcat

SEQ ID NO: 148 - DNA, 53 bp, synthetic construct
  source 1..53: mol_type = other DNA
agtggttgtggtgtgacagttggcacaggatttacatcatcatacatcatcat

SEQ ID NO: 149 - DNA, 55 bp, synthetic construct
  source 1..55: mol_type = other DNA
ggctcaccctcagcatttctacgccataaggttttacatcatcatacatcatcat

SEQ ID NO: 150 - DNA, 56 bp, synthetic construct
  source 1..56: mol_type = other DNA
agtttcatatagagcccgcaagcattgcacacatttacatcatcatacatcatcat

SEQ ID NO: 151 - DNA, 54 bp, synthetic construct
  source 1..54: mol_type = other DNA
gtaggagtcatagggacagagccactgctgttttacatcatcatacatcatcat

SEQ ID NO: 152 - DNA, 60 bp, synthetic construct
  source 1..60: mol_type = other DNA
cctgaggtggtcgcttgtgtagaaggagaagtatttttttacatcatcatacatcatcat

SEQ ID NO: 153 - DNA, 55 bp, synthetic construct
  source 1..55: mol_type = other DNA
ggcgttttctcccactgcagacatcactgatgtttacatcatcatacatcatcat

SEQ ID NO: 154 - DNA, 56 bp, synthetic construct
  source 1..56: mol_type = other DNA
acctgaatacttgaggtcactgttctcggggtttttacatcatcatacatcatcat

SEQ ID NO: 155 - DNA, 56 bp, synthetic construct
  source 1..56: mol_type = other DNA
ggacagactgacacctatgtagaggccgtcttgtttacatcatcatacatcatcat

SEQ ID NO: 156 - DNA, 53 bp, synthetic construct
  source 1..53: mol_type = other DNA
gtcgcacggaggatgtgacttcggcaggggtttacatcatcatacatcatcat

SEQ ID NO: 157 - DNA, 53 bp, synthetic construct
  source 1..53: mol_type = other DNA
aggccagggccagagcacaccaagaatccttttacatcatcatacatcatcat

SEQ ID NO: 158 - DNA, 53 bp, synthetic construct
  source 1..53: mol_type = other DNA
gccctccttgcctcttggtagcaccagctctttacatcatcatacatcatcat

SEQ ID NO: 159 - DNA, 60 bp, synthetic construct
  source 1..60: mol_type = other DNA
tggacaatatcagacacaagtggtatgaggccttcagtttacatcatcatacatcatcat

SEQ ID NO: 160 - DNA, 60 bp, synthetic construct
  source 1..60: mol_type = other DNA
gcacagaaatcacgcatcgaaggaatgttatgtctgctttacatcatcatacatcatcat

SEQ ID NO: 161 - DNA, 50 bp, synthetic construct
  source 1..50: mol_type = other DNA
acatcatcatgggccttttggcccatgatgatgtatgatgatgttttttt

SEQ ID NO: 162 - DNA, 43 bp, synthetic construct
  source 1..43: mol_type = other DNA
ccccgaaagtggcctcgggccttttggcccgaggccactttcg

SEQ ID NO: 163 - DNA, 29 bp, synthetic construct
  source 1..29: mol_type = other DNA
ttatgatgatgtatgatgtatgatgatgt

SEQ ID NO: 164 - DNA, 20 bp, synthetic construct
  source 1..20: mol_type = other DNA
tcgagacgccctagcgggaa

SEQ ID NO: 165 - DNA, 20 bp, synthetic construct
  source 1..20: mol_type = other DNA
gaacgcgctaggtacctgga

SEQ ID NO: 166 - DNA, 20 bp, synthetic construct
  source 1..20: mol_type = other DNA
caaggggtctttaaacctcc

SEQ ID NO: 167 - DNA, 20 bp, synthetic construct
  source 1..20: mol_type = other DNA
gtcggaaggtttcacaccac

SEQ ID NO: 168 - DNA, 20 bp, synthetic construct
  source 1..20: mol_type = other DNA
cagcaaacgggaccggactc

SEQ ID NO: 169 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 170 - RNA, 330 nt, Homo sapiens
  source 1..330: mol_type = other RNA
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactag

SEQ ID NO: 171 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 172 - RNA, 1788 nt, Homo sapiens
  source 1..1788: mol_type = other RNA
atggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctga

(19) United States
(12) Patent Application Publication
(10) Pub. No.: US 2024/0384353 A1
(43) Pub. Date: Nov. 21, 2024

(54) METHODS OF TREATING PANCREATIC CANCER

(71) Applicants: Fred Hutchinson Cancer Center, Seattle, WA (US); University of Washington, Seattle, WA (US)

(72) Inventors: Sita Kugel, Seattle, WA (US); Brian Beliveau, Seattle, WA (US); Naomi Yamamoto, Seattle, WA (US); Stephanie Dobersch, Seattle, WA (US)

(73) Assignees: Fred Hutchinson Cancer Center, Seattle, WA (US); University of Washington, Seattle, WA (US)

(21) Appl. No.: 18/607,269

(22) Filed: Mar. 15, 2024

Related U.S. Application Data
(60) Provisional application No. 63/490,498, filed on Mar. 15, 2023.

(51) Int. Cl. C12Q 1/6886; A61K 31/337; A61K 31/7068; A61K 47/64; A61P 35/00

(52) U.S. Cl. CPC C12Q 1/6886; A61K 31/337; A61K 31/7068; A61K 47/643; A61P 35/00; C12Q 2600/106; C12Q 2600/158

(57) ABSTRACT

Provided herein are biomarkers, methods of diagnosing, methods of treatment, methods of monitoring treatment, and methods of selecting treatment in a subject suspected of, or suffering from, Pancreatic cancer. In some embodiments, the methods disclosed herein comprise determining expression levels of at least one biomarker in one or more biological sample obtained from the subject, and comparing the expression levels of the at least one biomarker in the one or more biological sample obtained from the subject with an expression level of the at least one biomarker in a reference/control sample. In some embodiments, the at least one biomarker comprises High mobility Group A2 (HMGA2). In some embodiments, the pancreatic cancer is Pancreatic Ductal Adenocarcinoma (PDA). Also provided herein are reagents, compositions, and kits for the detection, diagnosis, and prognosis of pancreatic cancer.

### CROSS-REFERENCE TO RELATED APPLICATIONS

This application claims the benefit of U.S. Provisional Application No. 63/490,498, filed Mar. 15, 2023, the disclosure of which is hereby expressly incorporated by reference herein in its entirety.

### STATEMENT OF GOVERNMENT LICENSE RIGHTS

This invention was made with Government support under CA241472 awarded by the National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P81US_Seq_List_20240315.xml. The XML file is 160,335 bytes; was created on Mar. 15, 2024; and is being submitted electronically via Patent Center with the filing of the specification.

### BACKGROUND

Pancreatic Ductal Adenocarcinoma (PDA) is an extremely lethal disease with a 5-year survival rate of less than 10%. Numerous phase 3 trials of agents effective in other malignancies have failed to benefit unselected PDA populations, although patients do occasionally respond. Studies in other solid tumors have shown that heterogeneity in response is determined, in part, by molecular differences between tumors. Furthermore, treatment outcomes are improved by targeting drugs to tumor subtypes in which they are selectively effective, with breast and lung cancers providing recent examples.

However, identification of PDA molecular subtypes has been frustrated by a paucity of tumor specimens as well as the low cellularity and stromal richness of PDA tumors, leading to much debate regarding the value of existing methods for subtyping PDA. Current methods classify PDA into two principal subtypes based on transcriptional signatures: classical and basal. Although these molecular subtypes of PDA may theoretically provide new insights for precision medicine approaches, there is still no consensus on practical application of the subtype classification for clinical decision-making or for prognosis in PDA.

Basal-type PDA is the more aggressive and deadly form of PDA, makes up approximately 25% of pancreatic tumors, and has the worst overall prognosis of all subtypes of pancreatic cancer. The poor survival rate has improved only modestly over the years because of the lack of effective chemotherapeutic strategies for PDA. Standard of care for patients with metastatic pancreatic cancer with good performance status are combination chemotherapy with FOLFIRINOX (FFX) (folinic acid, 5-fluorouracil, irinotecan and oxaliplatin) or nab-paclitaxel plus Gemcitabine (GA) with median survival of less than 1 year. The selection of these therapies relies solely on patient performance status and comorbidities. However, the response of individual patients to treatment is variable. Consequently, the therapeutic strategies for PDA are lacking and remain far behind those in other solid tumors, in which biomarker selection for targeted therapies has dramatically improved treatment approaches and patient prognosis. Therefore, the development of biomarkers that allow for selection and identification of targeted therapies for individual patients with PDA is critical.

Accordingly, a need exists for robust biomarkers, methods, reagents, systems, and kits that enable identification of therapeutic vulnerabilities in PDA patients that may be exploited in the clinic to tailor therapeutic intervention in an informed manner.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Embodiments disclosed herein concern methods, compositions, and kits related to assessing, prognosis, and/or treating Pancreatic Ductal Adenocarcinoma.

In some embodiments, provided herein is a method of treating a subject identified as suffering from Pancreatic Ductal Adenocarcinoma. In some embodiments, the method comprises administering to the subject an effective amount of at least one therapeutic compound as first-line treatment. In an embodiment, the subject is identified as suffering from PDA by a method comprising: (i) obtaining one or more biological sample from the subject; (ii) determining/quantifying/measuring expression levels of at least one biomarker in the one or more biological sample obtained from the subject; and (iii) comparing the expression levels of the at least one biomarker in the one or more biological sample obtained from the subject with expression level of the at least one biomarker in a reference/control sample. In an embodiment, a differential expression of the at least one biomarker in the biological sample of the subject relative to the expression of the biomarker in the reference/control sample identifies the subject as suffering from PDA. In some embodiments, the at least one biomarker comprises High mobility Group A2 (HMGA2). In an embodiment, the differential expression comprises an overexpression of HMGA2 (HMGA2^{high}) in the one or more biological sample relative to the reference/control sample. In some embodiments, the at least one therapeutic compound comprises FOLFIRINOX. In some embodiments, the differential expression comprises a lower expression of HMGA2 (HMGA2^{low}) in the one or more biological sample relative to the reference/control sample. In some embodiments, the at least one therapeutic compound comprises Gemcitabine.

In an embodiment, the method further comprises determining/measuring/quantifying the expression of at least one other biomarker in the biological sample obtained from the subject and comparing the expression levels of the at least one other biomarker in the one or more biological sample obtained from the subject with expression level of the at least one other biomarker in a reference/control sample. In an embodiment, the at least one other biomarker comprises GATA binding protein 6 (GATA6). In some embodiments, the differential expression comprises an overexpression of HMGA2 (HMGA2^{high}) and a lower expression of GATA6 (GATA6^{low}) relative to the expression of HMGA2 and GATA6, respectively, in a reference/control sample. In some embodiments, the at least one therapeutic compound comprises FOLFIRINOX (FFX). In some embodiments, the differential expression comprises a lower expression of HMGA2 (HMGA2^{low}) and an overexpression of GATA6 (GATA6^{high}) relative to the expression of HMGA2 and GATA6, respectively, in a reference/control sample. In some embodiments, the at least one therapeutic compound comprises Gemcitabine.

In some embodiments, the reference sample is a biological sample obtained from a healthy subject not suffering from PDA. In an embodiment, the reference sample is a biological sample corresponding to the biological sample obtained from the subject suffering from PDA. In some embodiments, the one or more biological sample comprises a biopsy tissue or resected tissue of the Pancreatic Ductal Adenocarcinoma.

In some embodiments, the methods disclosed herein are effective in increasing overall median survival in the subject suffering from PDA.

Provided herein is a method of treating a subject suffering from Pancreatic Ductal Adenocarcinoma, the method comprising: administering to the subject an effective amount of FOLFIRINOX as first line of treatment. In some embodiments, the subject has been identified as suffering from basal subtype of PDA by a method comprising the steps of: obtaining one or more biological sample from the subject suffering from PDA; determining/quantifying/measuring expression levels of at least one biomarker in the one or more biological sample obtained from the subject; comparing the expression levels of the at least one biomarker in the one or more biological sample obtained from the subject with an expression level of the at least one biomarker in a reference/control sample; and identifying the subject as having a basal subtype of Pancreatic Ductal Adenocarcinoma when the at least one biomarker is differentially expressed in the biological sample obtained from the subject relative to the expression levels of the biomarker in the reference or control sample. In some embodiments, the at least one biomarker comprises High mobility Group A2 (HMGA2). In an embodiment, the differential expression comprises an overexpression of HMGA2 in the one or more biological sample obtained from the subject relative to the expression of the biomarker in the reference/control sample.

In some embodiments, the method further comprises determining expression levels of at least one other biomarker in one the one or more biological samples obtained from the subject. In some embodiments, the at least one other biomarker comprises GATA6. In an embodiment, the differential expression comprises a lower GATA6 expression in the one or more biological sample relative to the expression of GATA6 in the reference/control sample. In some embodiments, the differential expression comprises an overexpression of HMGA2 (HMGA2^{high}) and a lower expression of GATA6 (GATA6^{low}) in the one or more biological sample obtained from the subject relative to the expression level of HMGA2 and GATA6, respectively, in the reference/control sample.

The present disclosure further provides a method of treating a subject suffering from Pancreatic Ductal Adenocarcinoma, the method comprising: administering to the subject an effective amount of Gemcitabine as first line of treatment. In some embodiments, the subject has been identified as suffering from classical subtype of PDA by a method comprising the steps of: obtaining one or more biological sample from the subject suffering from PDA; determining/quantifying/measuring expression levels of at least one biomarker in the one or more biological sample obtained from the subject; comparing the expression levels of the at least one biomarker in the one or more biological sample obtained from the subject with an expression level of the at least one biomarker in a reference/control sample; and identifying the subject as having a classical subtype of Pancreatic Ductal Adenocarcinoma when the at least one biomarker is differentially expressed in the biological sample obtained from the subject relative to the expression levels of the biomarker in the reference or control sample. In an embodiment, the at least one biomarker comprises High mobility Group A2 (HMGA2). In some embodiments, the differential expression comprises a lower expression of HMGA2 in the one or more biological sample of the subject relative to the expression of the biomarker in the reference/control sample.

In some embodiments, the method further comprises determining expression levels of at least one other biomarker in the one or more biological samples obtained from the subject. In an embodiment, the at least one other biomarker comprises GATA6. In some embodiments, the differential expression comprises an overexpression of GATA6 in the one or more biological sample relative to the expression of GATA6 in the reference/control sample. In an embodiment, the differential expression comprises an overexpression of GATA6 (GATA6^{high}) and a lower expression of HMGA2 (HMGA2^{low}) in the one or more biological sample obtained from the subject relative to the expression level of HMGA2 and GATA6, respectively, in the reference/control sample.

Also provided herein is a method of increasing overall median survival in a subject suffering from Pancreatic Ductal Adenocarcinoma. In some embodiments, the method comprises administering to the subject an effective amount of at least one PDA subtype specific therapeutic compound as first-line treatment. In some embodiments, the subject has been diagnosed with the PDA subtype by a method comprising the steps of: obtaining one or more biological sample from the subject; determining/quantifying/measuring expression levels of at least one biomarker in the one or more biological sample obtained from the subject; and comparing the expression levels of the at least one biomarker in the one or more biological sample obtained from the subject with expression level of the at least one biomarker in a reference/control sample. In some embodiments, a differential expression of the at least one biomarker in the biological sample of the subject relative to the expression of the biomarker in the reference/control sample identifies the subject as suffering from the subtype of PDA. In some embodiments, the at least one biomarker comprises High mobility Group A2 (HMGA2). In an embodiment, the differential expression comprises an overexpression of HMGA2 (HMGA2^{high}) in the one or more biological sample relative to the reference/control sample. In some embodiments, the at least one PDA subtype specific therapeutic compound administered as first-line treatment comprises FOLFIRINOX. In some embodiments, the subject is diagnosed with basal subtype of PDA. In some embodiments, the differential expression comprises a lower expression of HMGA2 (HMGA2^{low}) in the one or more biological sample relative to the reference/control sample. In an embodiment, the at least one PDA subtype specific therapeutic compound administered as first-line treatment comprises Gemcitabine. In some embodiments, the subject is diagnosed with classical subtype of PDA.

In some embodiments, the method further comprises determining/measuring/quantifying the expression of at least one other biomarker in the biological sample obtained from the subject and comparing the expression levels of the at least one other biomarker in the one or more biological sample obtained from the subject with expression level of the at least one other biomarker in a reference/control sample. In an embodiment, the at least one other biomarker comprises GATA binding protein 6 (GATA6). In some embodiments, the differential expression comprises an overexpression of HMGA2 (HMGA2^{high}) and a lower expression of GATA6 (GATA6^{low}) relative to the expression of HMGA2 and GATA6, respectively, in a reference/control sample. In some embodiments, the subject is diagnosed with basal subtype of PDA, and the at least one PDA subtype specific therapeutic compound administered as first-line treatment comprises FOLFIRINOX. In a related embodiment, the differential expression comprises a lower expression of HMGA2 (HMGA2^{low}) and an overexpression of GATA6 (GATA6^{high}) relative to the expression of HMGA2 and GATA6, respectively, in a reference/control sample. In some embodiments, the subject is diagnosed with classical subtype of PDA, and wherein the at least one therapeutic compound administered as first-line treatment comprises Gemcitabine.

### DESCRIPTION OF THE DRAWINGS

This patent or application file contains at least one drawing executed in color. Copies of this patent or patent application publication with color drawing(s) will be provided by the Office upon request and payment of the necessary fec.

The foregoing aspects and many of the attendant advantages of this invention will become more readily appreciated as the same become better understood by reference to the following detailed description, when taken in conjunction with the accompanying drawings, wherein:

FIG. 1 shows HMGA2 promotes the aggressive disease biology of basal PDA. A higher expression of HMGA2 in basal PDA cell lines is observed as compared to classical PDA cell lines as measured by Western blot with β-actin (as control);

FIGS. 2A-2B show HMGA2 is expressed at a higher level in basal PDA. FIG. 2A shows HMGA2 expression levels from RNA-seq data of human PDA samples classified as belonging to either basal/QM/squamous or classical/progenitor subtypes using the Moffit's (“tumor” and “stroma” classification), Collisson's, and Bailey's classifications; 236 subjects had advanced stage pancreas cancer, 139 patients received FOLFIRINOX (folinic acid, 5-fluorouracil, irinotecan and oxaliplatin), and 97 received first line therapy nab-paclitaxel plus Gemcitabine. Biospecimens underwent laser capture microscopy to improve tumor cellularity for genomic analyses and were subjected to whole-genome sequencing and RNA-seq. FIG. 2B shows HMGA2 is expressed at higher levels in basal PDA. Kruskal-Wallis test (compare all subgroups): FDR-adjusted p-value=1.586045×10⁻²⁰; Wilcoxon rank-sum test (basal vs. classical): FDR-adjusted p-value=2.965594×10⁻¹⁹;

FIG. 3 shows a survival curve of HMGA2^{high} expressing versus HMGA2^{low} expressing PDA patients with advanced disease treated with either FOLFIRINOX (FFX) (FIG. 3, left panel) or nab-paclitaxel plus Gemcitabine (FIG. 3, right panel). Response to chemotherapy for 236 advanced stage patients, out of which 139 patients received FFX and 97 received GA as first line therapy. HMGA2 expression in metastatic and resectable patient samples from the COMPASS trial is higher in the basal subtype. Samples were purified by laser capture microdissection before RNA sequencing. The HMGA2^{high} state/basal subtype predicted efficacy from GA. Patients with advanced disease and high versus low levels of HMGA2 had shorter overall survival (6.58 months for HMGA2^{high} vs 12 months for HMGA2^{low}, HR: 0.4152, p value: 0.0001) when treated with GA as first line therapy;

FIG. 4 shows the Cancer Genome Atlas (TCGA) Pancreatic Adenocarcinoma (PAAD) survival for HMGA2. Analyses of proteomic data from TCGA (The Cancer Genome Atlas) PDA samples indicated higher levels of HMGA2 correlated with a worse overall survival;

FIG. 5 shows HMGA2 immunohistochemistry (IHC) (left, brown) and negative IHC (right);

FIG. 6 shows HMGA2^{high} (left) and HMGA2^{low} (right) expression in formalin fixed paraffin embedded (FFPE) human PDA tissue, stained with hematoxylin and eosin, and is representative of the tissue microarray showing the PDA tumor tissue;

FIG. 7 shows HMGA2^{high} (left) and HMGA2^{low} (right) expression in formalin fixed paraffin embedded (FFPE) human PDA tissue, using IHC staining;

FIGS. 8A-8B show HMGA2^{high} (FIG. 8A, left panel) and HMGA2^{low} (FIG. 8A, right panel) expression in formalin fixed paraffin embedded (FFPE) human PDA tissue, using in situ hybridization (ISH) staining. HMGA2^{high} appears red. HMGA2^{low} appears green, FIG. 8B shows GATA6 (upper left and center panels), HMGA2 (upper right panel) as detection by RNA ISH in FFPE of human PDA cells grown as subcutaneous tumors. FIG. 8B bottom panels show the in-situ hybridization (ISH) for HMGA2 levels in formalin fixed paraffin embedded (FFPE) human PDA tissue. ITS nucleolar probe is a positive control (FIG. 8B, lower left and center panel) and negative control (FIG. 8B, lower right panel);

FIGS. 9A-9B show HMGA2 and GATA6 status predict survival. Expression levels of HMGA2 and GATA6 were determined by immunohistochemistry in Tissue microarray comprising human PDA tumors from over 800 patients, along with matched (normal pancreatic tissue) controls correlated to clinical data (stage at diagnosis, treatment history) stained and scored for HMGA2 (FIG. 9A) and GATA6 (FIG. 9B);

FIG. 10 shows overall probability of median survival post-surgery based on expression levels of both HMGA2 and GATA6 identified four separate groupings based on HMGA2 and GATA6 expression levels with HMGA2^{high}/GATA6^{low} showing the poorest overall median survival and HMGA2^{low}/GATA6^{high} showing the best overall median survival. Expression of the biomarkers was assessed by immunohistochemistry in Tissue microarray comprising human PDA tumors from over 800 patients, along with matched (normal pancreatic tissue) controls correlated to clinical data (stage at diagnosis, treatment history) stained and scored for HMGA2 and GATA6 (FIG. 10);

FIGS. 11A-11B show using both HMGA2 and GATA6 as biomarkers allows for accurate prediction of response to overall median survival in Caucasian (FIG. 11A) and black populations (FIG. 11B). Expression of the biomarkers was assessed by immunohistochemistry in Tissue microarray comprising human PDA tumors from over 800 patients, along with matched (normal pancreatic tissue) controls correlated to clinical data (stage at diagnosis, treatment history) stained and scored for HMGA2 and GATA6;

FIGS. 12A-12B show HMGA2/GATA6 predict prognosis to Gemcitabine treatment. HMGA2^{high}/GATA6^{low} expressing tumors show an overall low median survival when treated with Gemcitabine as first-line therapy as compared to HMGA2^{low}/GATA6^{high} tumors treated with Gemcitabine as first-line therapy (FIG. 12A). HMGA2^{high}/GATA6^{low} expressing tumors show an overall low median survival when treated with Gemcitabine/5 FU as first-line therapy as compared to HMGA2^{low}/GATA6^{high} tumors treated with Gemcitabine/5 FU as first-line therapy (FIG. 12B).

### DETAILED DESCRIPTION

Reference is made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein which could be used in, and are within, the scope of the practice of the present invention. The present invention is in no way limited to the methods and materials described.

Unless defined otherwise, technical, and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including.” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. “Biological sample” includes sections of tissues such as biopsy and autopsy samples, and frozen sections taken for histologic purposes. Such samples include pancreatic cancer tissues, cultured cells, e.g., primary cultures, explants, and transformed cells. A biological sample is typically obtained from a mammal, such as a primate, e.g., human. The biological sample, in some embodiments, may include metastatic tissue. It would be readily understood by those skilled in the art that expression levels of biomarkers disclosed herein could be measured in biological samples such as tissue samples obtained by fine needle aspiration (FNAB) (a preferred embodiment), formalin-fixed paraffin embedded (FFPE) tissue, tissue microarrays (TMA), fresh-frozen or freshly obtained pancreatic cancer biopsy material.

A “biopsy” refers to the process of removing a tissue sample for diagnostic or prognostic evaluation, and to the tissue specimen itself. Any biopsy technique known in the art can be applied to the diagnostic and prognostic methods. The biopsy technique applied will depend on the tissue type to be evaluated, the size and type of the tumor, among other factors. Representative biopsy techniques include, but are not limited to, excisional biopsy, incisional biopsy, needle biopsy, and surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. Biopsy techniques are discussed, for example, in Harrison's Principles of Internal Medicine, Kasper et al. editors, McGraw Hill, 2005. Obtaining a biopsy includes both direct and indirect methods, including obtaining the biopsy from the patient or obtaining the biopsy sample after it is removed from the patient.

As used herein, “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

As used herein, “marker” and “biomarker” are used interchangeably to refer to a target molecule that indicates or is a sign of a normal or abnormal process in an individual/subject or of a disease or other condition in an individual/subject. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The terms “level” or “levels” with reference to one or more biomarker is meant to encompass a score, quantitative measurement, a qualitative assessment, or other acceptable observation obtained when a biomarker or observation correlated to a biomarker is assessed. The exact nature of the “value” or “level” depends on the specific design and components of the particular analytical method employed to detect the biomarker.

The terms “overexpress”, “overexpression”, “overexpressed”, “up-regulate”, “high”, or “up-regulated” interchangeably refer to a biomarker that is transcribed or translated at a detectably greater level, usually in a cancer cell, in comparison or relative to a non-cancer cell or cancer cell (from a control or reference sample) that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis. Overexpression can be detected using conventional techniques for detecting mRNA (i.e., RT-PCR, PCR, hybridization) or proteins (i.e., ELISA, immunohistochemical techniques, mass spectroscopy). Overexpression can be 10%, 20%, 30%, 40%, 50%, 60%, 70%, 80%, 90% or more (or any range derivable therein) in comparison to a reference/control sample, normal cell or cancer cell that is not associated with the worst or poorest prognosis. In certain instances, overexpression is 1-fold, 2-fold, 3-fold, 4-fold 5, 6, 7, 8, 9, 10, or 15-fold or more higher levels of transcription or translation (or any range derivable therein) in comparison to a reference/control sample, non-cancer cell or cancer cell that is not associated with the worst or poorest prognosis.

The term “lower expression” or “low expression” or “low” includes a lower expression of the biomarker due to transcription, post transcriptional processing, translation, post-translational processing, cellular localization, and/or RNA and protein stability, as compared to a non-cancer cell or cancer cell that is not associated with the worst or poorest prognosis. A lower expression can be detected using conventional techniques for detecting mRNA (i.e., RT-PCR, PCR, hybridization) or proteins (i.e., ELISA, immunohistochemical techniques, mass spectroscopy). A lower expression can be 10%, 20%, 30%, 40%, 50%, 60%, 70%, 80%, 90% or more (or any range derivable therein) in comparison to a reference/control sample, normal cell or cancer cell that is not associated with the worst or poorest prognosis. In certain instances, lower expression is 1-fold, 2-fold, 3-fold, 4-fold 5, 6, 7, 8, 9, 10, or 15-fold or more lower levels of transcription or translation (or any range derivable therein) in comparison to a reference/control sample, non-cancer cell or cancer cell that is not associated with the worst or poorest prognosis.

The comparison may be a direct comparison where the expression level of a control is measured at the same time as the biological sample obtained from the subject or it may be a level of expression that is determined from a previously evaluated sample or an average of levels of expression of previously evaluated sample(s). Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level or may be subject to alternative splicing to result in a different polypeptide product. Such differences may be evidenced by a variety of changes including mRNA levels, surface expression, secretion, or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “subject” or “individual” or “patient”, refers to a test subject or patient. The subject can be a mammal or a non-mammal. In various embodiments, the subject is a mammal. A mammalian subject can be a human or non-human. In various embodiments, the subject is a human. A healthy or normal subject is an individual in which the disease or condition of interest (including, for example, pancreatic cancer, pancreatic-associated diseases, or other pancreatic conditions) is not detectable by conventional diagnostic methods.

“Diagnose”, “diagnosing”, “diagnosis”, and variations thereof refer to the detection, determination, or recognition of a health status or condition of an individual or subject on the basis of one or more signs, symptoms, data, or other information pertaining to that individual. The health status of an individual or subject can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, and the like, encompass, with respect to a particular disease or condition, the initial detection of the disease; the characterization or classification of the disease; the detection of the progression, remission, or recurrence of the disease; and the detection of disease response after the administration of a treatment or therapy to the individual.

“Prognosis” generally refers to a prediction of the probable course or outcome of the disease. As used herein, prognosis includes the forecast or prediction of any one or more of the following: duration of survival of a subject susceptible to or diagnosed with a cancer or disease, duration of recurrence-free survival, duration of progression free survival of a subject susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

Provided herein are biomarkers, methods of diagnosing, methods of treatment, methods of monitoring treatment, and methods of selecting treatment in a subject suspected of or suffering from Pancreatic cancer. In some embodiments, the pancreatic cancer is Pancreatic Ductal Adenocarcinoma. In some embodiments, the subject is suspected of or is suffering from classical PDA. In some embodiments, the subject is suspected of or is suffering from basal PDA. Also provided herein are reagents, compositions, and kits for the detection, diagnosis, and prognosis of pancreatic cancer.

Pancreatic ductal adenocarcinoma is among the most lethal cancers. Known risk factors for this disease are currently insufficient in predicting mortality. Currently the only curative option for PDA is surgical resection (pancreaticoduodenectomy), however less than 20% of patients have resectable tumor due to the aggressiveness of the disease. Combination chemotherapy is used in the treatment of most patients with advanced PDA, yet the field is lacking robust biomarkers of outcome to guide regimen selection. Standard therapy for PDA is Gemcitabine and Gemcitabine combinations with other drugs. In recent years FOLFIRINOX and targeted EGFR inhibition by erlotinib combined with Gemcitabine showed only modest improvements in response rates and overall survival. PDA is almost unique in promoting an excess production of other components of the stroma, resulting in a complex tumor microenvironment that contributes to tumor development, progression, and response to treatment.

Currently, using transcriptome driven subtyping, PDA can be subtyped as: Classical or Basal, with strong alignment of each with potential clinical impact. However, the subtyping of PDA relies on techniques like gene expression microarrays and single-cell RNA sequencing to determine a transcriptional signature of several genes for each subtype. Given the highly acellular and stroma-rich nature of PDA, such techniques require microdissection of the tumor tissue from the stromal tissue to generate useful information thus adding another layer of complexity and in general are cumbersome, require more reporting time, and expensive. Thus, in contrast to other cancer types like colon and breast for which multiple molecular tests are available for risk prediction and/or molecular subtyping, such as PAM50, MammaPrint, Oncotype Dx Breast, Oncotype Dx Colon, there are limited number of molecular signatures/biomarkers defined for prognostication of pancreatic cancer, that are less cumbersome, less expensive, less time consuming and easy to perform than presently used transcriptional profiling. Importantly, none are available to guide clinical therapeutic decisions in practice. The present disclosure fulfills this unmet need in the art. Specifically, the present disclosure provides methods of using biomarkers for diagnosing and classifying subtypes of Pancreatic Ductal Adenocarcinoma, assessing the prognosis of PDA, or developing and/or selecting treatments for PDA based on detecting expression levels of one or more biomarkers disclosed herein. The biomarkers disclosed herein could be useful in tests, assays, or kits.

High mobility group protein A2 (HMGA2) is a small non-histone chromosomal protein:

(SEQ ID NO: 169)
MSARGEGAGQPSTSAQGQPAAPAPQKRGRGRPRKQQQEPTGEPSPKRPR
RPKGSKNKSPSKAAQKKAEATGEKRPRGRPRKWPQQVVQKKPAQEETEE
TSSQESAEED (human)
(SEQ ID NO: 170)
atgagcgcacgcggtgagggcgcggggcagccgtccacttcagcccagg
gacaacctgccgccccagcgcctcagaagagaggacgcggccgccccag
gaagcagcagcaagaaccaaccggtgagccctctcctaagagacccagg
ggaagacccaaaggcagcaaaaacaagagtccctctaaagcagctcaaa
agaaagcagaagccactggagaaaaacggccaagaggcagacctaggaa
atggccacaacaagttgttcagaagaagcctgctcaggaggaaactgaa
gagacatcctcacaagagtctgccgaagaggactag

It has no intrinsic transcriptional activity but can modulate transcription by altering chromatin architecture. HMGA2 binds to linker DNA in the chromatin and displaces histone H1 leading to chromatin decompaction. HMGA2 enhances the binding of chromatin remodeling complexes (CRC) that displace core histones. This facilitates assembly of RNA Pol II and transcription factors for initiation of transcription. Normally, HMGA2 protein is highly expressed in embryogenesis, while its expression is almost undetectable in most adult and differentiated tissues. HMGA2 is thought to play a fundamental role in the maintenance of stemness and in the regulation of differentiation.

Disclosed herein is the discovery that High mobility group (HMG) protein A2 (HMGA2) expression levels in PDA correlate with different subtypes of PDA, and therefore provide a useful biomarker for identifying/subtyping of PDA. Specifically, PDA tumors of a subject overexpressing HMGA2 (HMGA2^{high}) relative to expression levels of HMGA2 in a reference or control sample correlate with the more aggressive phenotype of basal PDA subtype and a low expression of HMGA (HMGA^{low}) in PDA tumors relative to expression level of HMGA2 in a reference or control sample correlates with the less aggressive phenotype of classical PDA subtype. Moreover, high levels of HMGA2 in PDA tumors correlates with poor survival as opposed to low levels of HMGA2. Further, HMGA2 expression is a useful predictor of sensitivity to nab-paclitaxel plus Gemcitabine treatment.

Accordingly, in an embodiment, the present disclosure provides a method of diagnosing Pancreatic Ductal Adenocarcinoma in a subject, the method comprising: (i) obtaining one or more biological sample from the subject;
(ii) determining/quantifying/measuring expression levels of at least one biomarker in the one or more biological sample obtained from the subject; and (iii) comparing the expression levels of the at least one biomarker in the one or more biological sample obtained from the subject with an expression level of the at least one biomarker in a reference/control sample; and determining/diagnosing the subject as having Pancreatic Ductal Adenocarcinoma when the expression levels of the at least one biomarker in the biological sample obtained from the subject is changed relative to the expression level of the biomarker in the reference or control sample.

In some embodiments, the at least one biomarker is High mobility group protein A2 (HMGA2). In some embodiments, HMGA2 is determined to be overexpressed (HMGA2^{high}) in the biological sample obtained from the subject relative to the expression levels of HMGA2 in the reference/control sample. In some embodiments, the overexpression of the HMGA2 (HMGA2^{high}) in the biological sample obtained from the subject relative to the expression level of HMGA2 in the reference sample diagnoses the subject as suffering from basal PDA. In some embodiments, the overexpression of the HMGA2 (HMGA2^{high}) in the biological sample obtained from the subject relative to the expression level of HMGA2 in the reference sample predicts poor overall survival. In some embodiments, the overexpression of the HMGA2 in the biological sample obtained from the subject relative to the expression level of HMGA2 in the reference sample classifies the subject as resistant to Gemcitabine-based treatment. In some embodiments, the overexpression of the HMGA2 in the biological sample obtained from the subject relative to the expression level of HMGA2 in the reference sample classifies the subject as sensitive to FOLFIRINOX-based treatment.

In some embodiments, HMGA2 is determined to be expressed at a lower level in the biological sample obtained from the subject relative to the reference sample (HMGA2^{low}). In some embodiments, the low expression level of HMGA2 in the biological sample relative to the reference sample diagnoses the subject as having classical PDA. In some embodiments, the low expression level of HMGA2 (HMGA2^{low}) in the biological sample obtained from the subject relative to the reference sample is predictive of better overall survival for the subject. In some embodiments, the low expression level of HMGA2 in the biological sample obtained from the subject relative to the reference sample classifies the subject as sensitive to Gemcitabine-based treatment.

The methods disclosed herein also contemplate determining/quantifying/measuring other known or novel biomarkers in combination with HMGA2 to diagnose, predict prognosis, monitor response to treatment/therapeutic agent, and/or to choose a subtype specific therapeutic agent or make treatment decisions based on validation and establishment of unique signature biomarkers using the methods disclosed herein.

Exemplary biomarkers that can be potentially used in combination with the determining/quantifying/measuring of HMGA2 expression levels include, but are not limited to, basal markers KRT17, KRT5, S100A2 and classical markers GATA binding protein 6 (GATA6), ECAD, CLDN18.2 and TTF1, and kirsten rat sarcoma viral oncogene homolog (KRAS).

Studies have demonstrated that PDA can arise from a variety of precursor cells by activating KRAS in distinct cellular compartments of the pancreas. It has also been demonstrated that cancer cell lines harboring mutant KRAS differ in their dependence on KRAS. These studies imply plasticity in either reliance on KRAS signaling or a cell-type specific role for mutant KRAS in different cells of origin/lineages in PDA, or both. These data further suggest that despite KRAS mutation in most PDAs, KRAS dependence might differ by PDA subtype.

Gene expression profiling, primarily in resected pancreatic tumors, describes a number of subtypes with considerable overlap, yet presently these do not inform clinical practice. However, surprisingly, the inventors have demonstrated that an RNA signature for HMGA2 in combination with GATA6 provides a robust signature that is useful in informing clinical decision and is of significant prognostic value.

GATA-family transcription factors are associated with tissue specific differentiation and have been demonstrated to be subtype specific markers in other cancers. GATA binding protein 6 (GATA6) is essential for pancreatic development and has been implicated in PDA. Specifically, GATA6 expression has been shown to align with the classical subtype and represents a surrogate marker for classical PDA. The gene signature associated with GATA6 overexpression (GATA6^{high}) relative to GATA6 expression in a control/reference sample is enriched in the classical subtype of PDA. Patients with tumors of a modified “basal-like” phenotype, or those with low GATA6 expression (GATA^{low}), have inferior outcomes compared with those with the “classical” phenotype. A low expression of GATA6 in basal-like tumors correlates with resistance to FOLFIRINOX.

In a related embodiment, the method further comprises determining/quantifying/measuring expression levels of at least one other biomarker in the biological sample obtained from the subject and comparing the expression level of the at least one other biomarker in the biological sample obtained from the subject with expression levels of the at least one other biomarker in the reference/control sample. In some embodiments, the at least one other biomarker is GATA6:

(SEQ ID NO: 171)
MALTDGGWCLPKRFGAAGADASDSRAFPAREPSTPPSPISSSSSSCSRG
GERGPGGASNCGTPQLDTEAAAGPPARSLLLSSYASHPFGAPHGPSAPG
VAGPGGNLSSWEDLLLFTDLDQAATASKLLWSSRGAKLSPFAPEQPEEM
YQTLAALSSQGPAAYDGAPGGFVHSAAAAAAAAAAASSPVYVPTTRVGS
MLPGLPYHLQGSGSGPANHAGGAGAHPGWPQASADSPPYGSGGGAAGGG
AAGPGGAGSAAAHVSARFPYSPSPPMANGAAREPGGYAAAGSGGAGGVS
GGGSSLAAMGGREPQYSSLSAARPLNGTYHHHHHHHHHHPSPYSPYVGA
PLTPAWPAGPFETPVLHSLQSRAGAPLPVPRGPSADLLEDLSESRECVN
CGSIQTPLWRRDGTGHYLCNACGLYSKMNGLSRPLIKPQKRVPSSRRLG
LSCANCHTTTTTLWRRNAEGEPVCNACGLYMKLHGVPRPLAMKKEGIQT
RKRKPKNINKSKTCSGNSNNSIPMTPTSTSSNSDDCSKNTSPTTQPTAS
GAGAPVMTGAGESTNPENSELKYSGQDGLYIGVSLASPAEVTSSVRPDS
WCALALA (human)
(SEQ ID NO: 172)
atggccttgactgacggcggctggtgcttgccgaagcgcttcggggccg
cgggtgcggacgccagcgactccagagcctttccagcgcgggagccctc
cacgccgccttcccccatctcttcctcgtcctcctcctgctcccggggc
ggagagcggggccccggcggcgccagcaactgcgggacgcctcagctcg
acacggaggcggcggccggacccccggcccgctcgctgctgctcagttc
ctacgcttcgcatcccttcggggctccccacggaccttcggcgcctggg
gtcgcgggccccgggggcaacctgtcgagctgggaggacttgctgctgt
tcactgacctcgaccaagccgcgaccgccagcaagctgctgtggtccag
ccgcggcgccaagctgagccccttcgcacccgagcagccggaggagatg
taccagaccctcgccgctctctccagccagggtccggccgcctacgacg
gcgcgcccggcggcttcgtgcactctgcggccgcggcggcagcagccgc
ggggggccagctccccggtctacgtgcccaccacccgcgtgggttccat
gctgcccggcctaccgtaccacctgcaggggtcgggcagtgggccagcc
aaccacgcgggcggcgcgggcgcgcaccccggctggcctcaggcctcgg
ccgacagccctccatacggcagcggaggcggcgcggctggcggcggggc
cgcggggcctggcggcgctggctcagccgcggcgcacgtctcggcgcgc
ttcccctactctcccagcccgcccatggccaacggcgccgcgcgggagc
cgggaggctacgcggcggcgggcagtgggggcgcgggaggcgtgagcgg
cggcggcagtagcctggcggccatgggcggccgcgagccccagtacagc
tcgctgtcggccgcgcggccgctgaacgggacgtaccaccaccaccacc
accaccaccaccaccatccgagcccctactcgccctacgtgggggcgcc
actgacgcctgcctggcccgccggacccttcgagaccccggtgctgcac
agcctgcagagccgcgccggagccccgctcccggtgccccggggtccca
gtgcagacctgctggaggacctgtccgagagccgcgagtgcgtgaactg
cggctccatccagacgccgctgtggcgggggacggcaccggccactacc
tgtgcaacgcctgcgggctctacagcaagatgaacggcctcagccggcc
cctcatcaagccgcagaagcgcgtgccttcatcacggcggcttggattg
tcctgtgccaactgtcacaccacaactaccaccttatggcgcagaaacg
ccgagggtgaacccgtgtgcaatgcttgtggactctacatgaaactcca
tggggtgcccagaccacttgctatgaaaaaagagggaattcaaaccagg
aaacgaaaacctaagaacataaataaatcaaagacttgctctggtaata
gcaataattccattcccatgactccaacttccacctcttctaactcaga
tgattgcagcaaaaatacttcccccacaacacaacctacagcctcaggg
gggggccccggtgatgactggtgcgggagagagcaccaatcccgagaac
agcgagctcaagtattcgggtcaagatgggctctacataggcgtcagtc
tcgcctcgccggccgaagtcacgtcctccgtgcgaccggattcctggtg
cgccctggccctggcctga

In some embodiments, the biological sample obtained from the subject has an overexpression of HMGA2 (HMGA2^{high}) and a lower expression of GATA6 (GATA6^{low}) relative to the expression level of HMGA2 and GATA6, respectively, in the reference/control sample. In some embodiments, the overexpression of the HMGA2 and the lower expression of GATA6 in the biological sample obtained from the subject relative to the expression of HMGA2 and GATA6, respectively, in the reference sample diagnoses the subject as suffering from basal PDA. In some embodiments, the overexpression of the HMGA2 and the lower expression level of GATA6 in the biological sample obtained from the subject relative to the expression level of HMGA2 and GATA6, respectively, in the reference sample predicts poor overall survival for the subject. In some embodiments, the overexpression of the HMGA2 and the lower expression level of GATA6 in the biological sample obtained from the subject as compared to the expression levels of HMGA2 and GATA6, respectively, in the reference sample classifies the subject as resistant to Gemcitabine-based treatment. In some embodiments, the overexpression of the HMGA2 in the biological sample obtained from the subject relative to the expression level of HMGA2 in the reference sample classifies the subject as sensitive to FOLFIRINOX-based treatment.

In some embodiments, the biological sample obtained from the subject has a lower expression of HMGA2 (HMGA2^{low}) and an overexpression of GATA6 (GATA6^{high}) as compared to the expression levels of HMGA2 and GATA6, respectively, in the reference sample. In some embodiments, the lower expression of the HMGA2 and the overexpression of GATA6 in the biological sample obtained from the subject as compared to the expression levels of HMGA2 and GATA6, respectively, in the reference sample diagnoses the subject as suffering from classical PDA. In some embodiments, the lower expression of the HMGA2 and the overexpression of GATA6 in the biological sample obtained from the subject relative to the expression of HMGA2 and GATA6, respectively, in the reference sample predicts better overall survival for the subject. In some embodiments, the lower expression of the HMGA2 and the overexpression of GATA6 in the biological sample obtained from the subject as compared to the expression of HMGA2 and GATA6, respectively, in the reference sample classifies the subject as sensitive to Gemcitabine-based treatment.

The expression levels or levels of biomarkers disclosed herein can be determined at the transcript, and/or protein level.

In some embodiments, the disclosure provides for methods of characterizing or subtyping a biological sample obtained from a subject. In some embodiments, the subject is suffering from PDA. In an embodiment, the method comprises determining the level of HMGA2 in the biological sample obtained from the subject. In some embodiments, determining the level of HMGA2 comprises determining the expression level of the HMGA2 protein or the transcript of hmga2.

In an embodiment, characterizing or subtyping the biological sample comprises determining an HMGA2 level relative to HMGA2 level in a control/reference sample. In an embodiment, determining the HMGA2 level comprises characterizing the HMGA2 level as HMGA2^{high} or HMGA2^{low}.

Determining/measuring/quantifying HMGA2 level comprises determining an HMGA2 level of the subject in comparison with a control or reference sample, or determining an HMGA2 level of the subject to have a particular value. Determining/measuring/quantifying HMGA2 level can comprise determining, measuring, quantifying HMGA2 protein levels or transcript levels.

In an exemplary embodiment, a determination of HMGA2^{high} comprises an HMGA2 level of about 25% of a measured/quantified HMGA2 level or higher relative to the measured/quantified levels of HMGA2 in the reference/control sample. A determination of HMGA2^{low} comprises HMGA2 level of about 25% of a measured/quantified HMGA2 expression levels or lower relative to the reference/control sample.

In some embodiments, characterizing or subtyping the biological sample comprises characterizing or subtyping the biological sample as basal PDA or classical PDA. In some embodiments, a determination of HMGA2^{high} characterizes or subtypes the PDA as basal PDA. In such an embodiment, a HMGA2^{high} characterization comprises administering to the subject an effective amount of FOLFIRINOX as a first-line treatment. In an embodiment, a basal characterization comprises not administering to the subject GA as a first-line treatment.

In some embodiments, a determination of HMGA2^{low} characterizes or subtypes the PDA as classical PDA. In an embodiment, a HMGA2^{low} characterization comprises administering to the subject an effective amount of FOLFIRINOX or GA. In such an embodiment, a HMGA2^{low} characterization comprises administering to the subject an effective amount of FOLFIRINOX or GA as a first-line treatment.

In an embodiment, a characterization of PDA as classical comprises administering to the subject FOLFIRINOX or GA. In such an embodiment, a characterization as classical comprises administering to the subject FOLFIRINOX or GA as a first-line treatment.

In some embodiments, the method of characterizing and/or subtyping the PDA comprises determining the level of at least one other biomarker. In some embodiments, the one other biomarker comprises GATA6. Determining the GATA6 level comprises determining GATA6 levels of the subject in comparison with a control or reference sample or determining an GATA6 level of the subject to have a particular value.

In an exemplary embodiment, a determination of GATA6^{high} comprises an HMGA2 level of about 25% of a measured/quantified GATA6 level or higher relative to the measured/quantified levels of GATA6 in the reference/control sample. A determination of GATA6^{low} comprises GATA6 level of about 25% of a measured/quantified GATA6 expression levels or lower relative to the reference/control sample.

In an embodiment, a determination of HMGA2^{high} and GATA6^{low} characterizes or subtypes or diagnoses the PDA as basal PDA. In such an embodiment, a HMGA2^{high} and GATA6^{low} characterization comprises administering to the subject an effective amount of FOLFIRINOX as a first-line treatment. In an embodiment, a basal characterization comprises not administering to the subject GA as a first-line treatment.

In an embodiment, a determination of HMGA2^{low} and GATA6^{high} characterizes or subtypes or diagnoses the PDA as classical PDA. In such an embodiment, a HMGA2^{low} and GATA6^{high} characterization comprises administering to the subject an effective amount of GA as a first-line treatment.

Certain embodiments are directed to methods of treating PDA based on the determination of the levels of the one or more biomarkers disclosed herein. In certain aspects, there may be provided methods for treating a subject determined to have cancer and with a predetermined expression profile of the one or more biomarkers disclosed herein.

In a further aspect, a determination of the levels of the one or more biomarkers and related systems that can establish a prognosis of a subject suffering from PDA can be used to identify subjects who may get benefit of a specific treatment modality. In the same way, those subjects who do not get much benefit from the specific treatment modality can be identified and can be offered alternative treatment(s).

In certain aspects, conventional therapy may be applied to a subject wherein the subject is identified or reported as having a good prognosis based on the assessment/determination of the levels of the one or more biomarkers disclosed herein. On the other hand, at least an alternative therapy may be prescribed, as used alone or in combination with conventional therapy, if a poor prognosis is determined by the disclosed methods, systems, or kits.

In some embodiments, the present disclosure provides a method of treating a subject identified as suffering from PDA, the method comprising: administering to the subject an effective amount of at least one therapeutic agent, wherein the subject is identified as suffering from PDA by a method comprising: (i) obtaining one or more biological sample from the subject suffering from PDA; (ii) determining/quantifying/measuring expression levels of at least one biomarker in the one or more biological sample obtained from the subject; and (iii) comparing the expression levels of the at least one biomarker in the one or more biological sample obtained from the subject with an expression level of the at least one biomarker in a reference/control sample, wherein a differential expression of the at least one biomarker in the biological sample of the subject relative to the expression of the biomarker in a reference/control sample identifies the subject as suffering from PDA. In some embodiments, the reference sample is a biological sample obtained from a healthy subject, wherein the healthy subject is a subject not suffering from or at risk for PDA. In some embodiments, the reference sample is a biological sample corresponding to the biological sample obtained from the subject.

In some embodiments, the at least one biomarker is High mobility group protein A2 (HMGA2). In some embodiments, HMGA2 is determined to be overexpressed (HMGA2^{high}) in the biological sample obtained from the subject relative to the expression levels of HMGA2 in the reference/control sample. In some embodiments, the overexpression of the HMGA2 in the biological sample obtained from the subject relative to the expression level of HMGA2 in the reference sample characterizes the subject as suffering from basal PDA. In such embodiments, the method comprises administering an effective amount of a therapeutic compound comprising FOLFIRINOX as first-line treatment. In some embodiments, the method is effective in increasing overall median survival in the subject. In an embodiment, the subject is resistant to Gemcitabine-based treatment.

In an alternative embodiment, HMGA2 is determined to be expressed at a lower level in the biological sample obtained from the subject relative to the reference sample (HMGA2^{low}). In some embodiments, the low expression level of HMGA2 in the biological sample relative to the reference sample characterizes the subject as having classical PDA. In such embodiments, the method comprises administering an effective amount of a therapeutic compound comprising Gemcitabine as first-line treatment. In some embodiments, the method is effective in increasing overall survival of the subject.

In some embodiments, the method further comprises determining/measuring expression levels of at least one other biomarker in the biological sample obtained from the subject and comparing the expression level of the at least one other biomarker in the biological sample obtained from the subject with expression levels of the at least one other biomarker in the reference/control sample. In some embodiments, the at least one other biomarker is GATA6.

In some embodiments, the biological sample obtained from the subject is determined to have an overexpression of HMGA2 (HMGA2^{high}) and a lower expression of GATA6 (GATA6^{low}) relative to the expression level of HMGA2 and GATA6, respectively, in the reference/control sample. In some embodiments, the HMGA2^{high} and GATA6^{low} in the biological sample obtained from the subject relative to the expression of HMGA2 and GATA6, respectively, in the reference sample identifies the subject as suffering from basal PDA. In such embodiments, the method comprises administering to the subject an effective amount of a therapeutic compound comprising FOLFIRINOX as first-line treatment. In some embodiments, the method is effective in increasing overall survival of the subject. In an embodiment, the subject is resistant to Gemcitabine-based treatment.

In some embodiments, the biological sample obtained from the subject is determined to have a lower expression of HMGA2 (HMGA2^{low}) and an overexpression of GATA6 (GATA6^{high}) relative to the expression level of HMGA2 and GATA6, respectively, in the reference/control sample. In some embodiments the HMGA2^{low} and GATA6^{high} in the biological sample obtained from the subject relative to the expression of HMGA2 and GATA6, respectively, in the reference sample identifies the subject as suffering from classical PDA. In such embodiments, the method comprises administering to the subject an effective amount of a therapeutic compound comprising Gemcitabine as a first-line of treatment. In some embodiments, the method is effective in increasing overall median survival in the subject.

In some embodiments, the present disclosure provides a method of treating a subject suffering from PDA, the method comprising: administering to the subject an effective amount of FOLFIRINOX as a first line of treatment. In some embodiments, the subject has been identified as suffering from basal subtype of PDA by a method comprising the steps of: (i) obtaining one or more biological sample from the subject suffering from PDA; (ii) determining/quantifying/measuring expression levels of at least one biomarker in the one or more biological sample obtained from the subject; (iii) comparing the expression levels of the at least one biomarker in the one or more biological sample obtained from the subject with an expression level of the at least one biomarker in a reference/control sample; and (iv) identifying the subject as having a basal subtype of Pancreatic Ductal Adenocarcinoma when the at least one biomarker is differentially expressed in the biological sample obtained from the subject relative to the expression levels of the biomarker in the reference or control sample. In some embodiments, the at least one biomarker comprises HMGA2 and the differential expression of HMGA2 comprises an overexpression (HMGA2^{high}) relative to level of HMGA2 the control/reference sample.

In some embodiments, the method of identifying the subject as suffering from basal subtype of PDA further comprises determining expression levels of at least one other biomarker in the one or more biological sample obtained from the subject. In some embodiments, the at least one other biomarker comprises GATA6. In some embodiments, the biological sample obtained from the subject has a lower GATA6 expression level (GATA low) relative to GATA6 expression levels in the reference/control sample. In some embodiments, the subject is identified as suffering from basal type of PDA when the biological sample obtained from the subject is determined to have an overexpression of HMGA2 (HMGA2^{high}) and a lower expression of GATA6 (GATA6^{low}) relative to the expression level of HMGA2 and GATA6, respectively, in the reference/control sample.

In some embodiments, the present disclosure provides a method of treating a subject suffering from PDA, the method comprising: administering to the subject an effective amount of Gemcitabine as first line of treatment. In some embodiments, the subject has been identified as suffering from classical subtype of PDA by a method comprising the steps of: (i) obtaining one or more biological sample from the subject suffering from PDA; (ii) determining/quantifying/measuring expression levels of at least one biomarker in the one or more biological sample obtained from the subject; (iii) comparing the expression levels of the at least one biomarker in the one or more biological sample obtained from the subject with an expression level of the at least one biomarker in a reference/control sample; and (iv) identifying the subject as having a classical subtype of Pancreatic Ductal Adenocarcinoma when the at least one biomarker is differentially expressed in the biological sample obtained from the subject relative to the expression levels of the biomarker in the reference or control sample. In some embodiments, the at least one biomarker comprises HMGA2 and the differential expression of HMGA2 comprises a lower expression level of HMGA2 (HMGA2^{low}) relative to level of HMGA2 the control/reference sample.

In some embodiments, the method of identifying the subject as suffering from classical subtype of PDA further comprises determining expression levels of at least one other biomarker in the one or more biological sample obtained from the subject. In some embodiments, the at least one other biomarker comprises GATA6. In some embodiments, the biological sample obtained from the subject has an overexpression of GATA6 (GATA^{high}) relative to GATA6 expression levels in the reference/control sample. In some embodiments, the subject is identified as suffering from classical subtype of PDA when the biological sample obtained from the subject is determined to have HMGA2^{low} and GATA6^{high} relative to the expression level of HMGA2 and GATA6, respectively, in the reference/control sample.

Also provided herein is a method of selecting a treatment option for a subject suffering from PDA, wherein the method comprises: identifying a subtype of PDA in the subject; and administering an effective amount of a subtype specific therapeutic compound as first-line treatment to the subject. In some embodiments, the subject is identified to be suffering from the basal subtype of PDA. In some embodiments, the subtype specific therapeutic compound comprises FOLFIRINOX (FFX). In an alternative embodiment, the subject is identified to be suffering from the classical subtype of PDA. In some embodiments, the subtype specific therapeutic compound comprises Gemcitabine.

The present disclosure also provides methods of increasing the overall median survival in a subject suffering from PDA, the method comprising: identifying/characterizing a subtype of PDA in the subject; and administering an effective amount of a subtype specific therapeutic compound to the subject.

“Treating” or “treatment,” as used herein, includes treatment of the disease or condition of interest in a mammal, preferably a human, having the disease or condition of interest, such as PDA, and includes: (a) inhibiting the disease or condition in a subject, i.e., arresting the disease or condition's development; (b) relieving (or ameliorating) the disease or condition, i.e., causing regression of the disease or condition; (c) relieving (or ameliorating) the symptoms resulting from the disease or condition, e.g., without addressing the underlying disease or condition; (d) preventing metastasis of the disease or condition; and/or (e) increasing or improving overall survival of the subject.

The term “therapeutically effective amount” or “an effective amount” refers to an amount of the drug or agent or a compound that may reduce the number of cancer cells; reduce the tumor size; inhibit (i.e., slow to some extent and particularly stop) cancer cell infiltration into peripheral organs; inhibit (i.e., slow to some extent and particularly stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy. The therapeutic agents disclosed herein may be combined with other therapeutic agents and/or modalities, such as surgery, radiation, immunotherapy, gene therapy, or a combination thereof.

As will be understood by those of ordinary skill in the art, the appropriate doses of the therapeutic agents/compositions disclosed herein will be approximately those already employed in clinical therapies wherein the therapeutic agent is administered alone or in combination with other agents or treatment modalities.

A method described herein may be performed alone or in conjunction with an additional therapy or therapeutic modality, such as chemotherapy, radiation therapy, surgery, hormone therapy, gene therapy, immunotherapy, chemoimmunotherapy, cryotherapy, ultrasound therapy, liver transplantation, local ablative therapy, radiofrequency ablation therapy, photodynamic therapy, and the like. The additional therapeutic modality may be administered before, after, sequentially, concurrently, or simultaneously with a method disclosed herein.

The therapeutic agents disclosed herein can be administered to an individual or subject (such as human) via various routes, including, for example, parenteral, intravenous, intraventricular, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the therapeutic agent may be used. In some embodiments, the therapeutic agent is administered intravenously. In some embodiments, the therapeutic agent is administered intraportally. In some embodiments, the therapeutic agent is administered intraarterially. In some embodiments, the therapeutic agent is administered intraperitoneally. In some embodiments, the therapeutic agent is administered intrathecally. In some embodiments, the therapeutic agent is administered through a ported catheter to spinal fluid. In some embodiments, the therapeutic agent is administered intraventricularly. In some embodiments, the therapeutic agent is administered systemically. In some embodiments, the therapeutic agent is administered by infusion. In some embodiments, the therapeutic agent is administered by infusion through an implanted pump. In some embodiments, the therapeutic agent is administered by a ventricular catheter. In some embodiments, the therapeutic agent is administered through a port or portacath. In some embodiments, the port or portacath is inserted into a vein (such as jugular vein, subclavian vein, or superior vena cava).

For comparison of the levels of biomarkers disclosed herein, a number of references or controls would be considered appropriate. For example, a biomarker level could be compared with the level of the biomarker in tissue known to be non-cancerous pancreatic tissue. A subject's own tissue could be used as a reference for comparison, or a population-derived value may be obtained. Local reference standards can be established if it is found that epidemiological variation exists among populations studied. Standards established within a group having a common demographic may also be established based on, for example, age, sex, smoking status, and other potentially influencing factors.

Thus, in some embodiments, the reference sample is a biological sample obtained from a healthy subject, wherein the healthy subject is a subject not suffering from PDA or diagnosed with PDA. In some embodiments, the reference sample is a biological sample corresponding to the biological sample obtained from the subject. The reference/control expression levels of the one or more biomarkers disclosed herein may be determined from a level of the one or more biomarkers in known non-cancerous pancreatic tissue or known pancreatic cancer tissue. The reference/control expression levels of the one or more biomarkers disclosed herein can also be determined from a level of the one or more biomarkers in the subject's own noncancerous tissue.

Statistical methods can be used to define the range of values for a reference/control. A range could be values within one standard deviation of the mean, and preferably values within two standard deviations of the mean. A level of biomarker within such a range or outside of the defined range may indicate that a tissue sample is the basal subtype of PDA versus classical subtype of PDA, respectively. An alternate reference for comparison of the biomarker level could be determined by establishing the level of biomarker in tissue known to be basal PDA. Statistical methods can be used to define the range of values for the alternate reference. A range could be values within one standard deviation of the mean, and preferably values within two standard deviations of the mean. A level of biomarker within this range would indicate that an unknown tissue is a basal subtype of PDA. A level of biomarker outside of this range would indicate that the unknown tissue is classical subtype of PDA.

Biomarker levels may be compared with a threshold level beyond which a positive indication is determined. For example, if a particular biomarker is shown to exhibit an increase (or decrease) relative to a reference/control, a threshold level of change, for example, a 5%, 10%, 15%, 20%, 25%, 30%, 35%, 40%, 45%, 50%, 75%, 100%, 125%, 150%, 200%, 300%, 400% or 500% increase, or a 5%, 10%, 15%, 20%, 25%, 30%, 35%, 40%, 45%, 50%, 75% or 100% decrease, relative to a reference/control may be set or pre-determined to allow a comparison from which a positive or negative indication can be derived.

Those of skill in the art are familiar with differentiating between significant up-regulation or down-regulation of expression of a biomarker relative to a reference or background expression of a biomarker. When a biomarker is up-regulated or overexpressed as an indication of a positive result, this can be easily distinguished from background (low-level values). Background expression levels may be used to form a “cut-off” above which increased staining will be scored as significant positive expression. Positive expression may be represented by a high level of antigen in tissue, or by a high proportion of cells from within a tissue that give a positive signal. When down-regulation of a biomarker is indicative of a positive result, those of skill in the art are also familiar with differentiating between significantly reduced expression of a biomarker, and background expression of a biomarker at a higher level. Indeed, background expression levels in such instances may be used to determine a “cut-off” below which decreased staining will be scored as lower expression. Lower expression may be represented by low levels of antigens in tissues, or alternatively, by a low proportion of cells from within a tissue that each give a low signal.

A biomarker can be determined to be statistically different from a reference standard or background if the mean or median expression level of the biomarker in a group forming a reference versus a group representing a positive diagnosis of PDA is to be statistically significant, as is the case in the present disclosure. Common tests for statistical significance include, among others, t-test, ANOVA₁ Kruskal-Wallis, Wilcoxon, Mann-Whitney and odds ration. Biological samples (of unknown status) can be compared with data from the reference group (negative control), and/or compared with data obtained from a positive control group known to have cancer.

As used herein, the phrases “indicative of” or “diagnosing a subtype of” Pancreatic Ductal Adenocarcinoma when referring to levels of a biomarker or an expression pattern of a biomarker which is diagnostic or confirmatory of disease such that the biomarker levels or expression pattern is found significantly more often in subjects with a disease than in subjects without the disease (as determined using routine statistical methods setting confidence levels at a typical minimum, such as at 95%). Preferably, an expression pattern which is indicative of disease is found in at least 70%, at least 75%, at least 80%, at least 85%, at least 90%, at least 95% or more in subjects who have the disease, and is found in less than 10%, less than 8%, less than 5%, less than 2.5%, or less than 1% of patients who do not have the disease. The phrase may also indicate an expression pattern which is diagnostic of disease such that the expression pattern more properly categorizes expression patterns of individuals/subjects with disease as compared with control expression patterns of individuals/subjects without disease using statistical algorithms for class prediction. Such comparisons would readily be understood by a person skilled in the art, and could be implemented using computerized means, such as for example commercially available programs available from Silicon Genetics (e.g., GeneSpring™).

A positive diagnosis could be based upon comparison of levels found in the population with respect to the expression that is seen in cancer compared to benign. Each biomarker may have a different scoring system and these variables can be evaluated both continuously and/or with different cut-off points to see what results in the best performance. A reference standard may easily be based on the expression by cancer and benign lesions seen in acceptable sized cohorts. Cut-off points to be utilized can be specified for each marker evaluated, based on population cohorts.

In one particular embodiment, the subtype of PDA can be diagnosed if the levels of the one or more biomarkers disclosed herein and being determined, in comparison to the reference, indicate, or on balance indicate, that the tissue sample is a subtype of PDA.

Biomarkers described herein can be used to support results and data obtained in conventional ways, such as by histopathology or cytomorphology examination of stained tissue sections. Should a conventionally derived diagnosis require confirmation, the panels of biomarkers described herein can provide such confirmation.

In one embodiment described herein, the prognosis of PDA can be observed and trends over time can be followed based on the one or more biomarkers disclosed herein. Such observations can be used to determine prognosis or outcome of a subject suffering from PDA. For example, the responsiveness to surgery, a type of therapy, or alternative treatment interventions may be observed and correlated to the one or more biomarkers described herein so that at the time a test assay is conducted, decisions can then be made as to the possible outcome for each method of treatment. Personalized treatment plans can be undertaken, depending on the outcome of the assessment of the at least one biomarker disclosed herein. In this way, patients/subjects and health care providers have more information on which to make treatment decisions, as well as personal decisions.

A skilled reader would also readily understand that the biomarkers disclosed herein may also useful for a) disease sub-classification of PDA lesions, b) determining the prognosis or outcome of a lesion/subject harboring a PDA lesion, c) predicting the response to surgery and/or other treatment intervention and/or d) as potential therapeutic targets alone or in combination to enable the development of targeted agents/drugs for treatment of PDA in general.

Expression levels or levels in a subject can be measured in cells of a biological sample obtained from the subject by methods known to those skilled in the art. For example, a tissue sample can be removed from a subject by conventional biopsy techniques. In another example, a body fluid sample, such as a lymph, blood or serum sample, or an exudate fluid sample such as a cancerous organ exudate may be used as the sample. A blood sample can be removed from the subject and white blood cells can be isolated for DNA extraction by standard techniques. The fluid or tissue sample obtained from the subject can be done prior to the initiation of radiotherapy, chemotherapy or other therapeutic treatment. A corresponding control tissue or blood sample can be obtained from unaffected or non-disease state tissues of the subject, from a normal (non-disease or non-cancerous) subject or population of normal subjects, or from cultured cells corresponding to the majority of cells in the subject's sample. The control tissue or blood sample is then processed along with the sample from the subject, so that the levels of expression in cells from the subject's sample can be compared to the corresponding expression levels from cells of the control sample.

The level of a gene product in a sample can be measured using any technique that is suitable for detecting RNA expression levels in a biological sample. Suitable techniques for determining RNA expression levels in cells from a biological sample are well known to those of skill in the art, including, for example, Northern blot analysis, RT-PCR, in situ hybridization. In one embodiment, the level of gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labelled DNA or RNA probes complementary to the RNA in question. Sec, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Some of the most important characteristics required for a diagnostic test to have clinical utility are case of usage, reproducibility, and reliability. A variety of methodologies can be utilized to assess, quantify expression levels and/or expression patterns of the one or more biomarkers disclosed herein in fine needle aspiration biopsy specimens and/or other biological samples or tissues obtained from a subject. In some embodiments, assessing levels and/or expression levels can comprise assessing and/or measuring/quantifying levels of DNA, transcript, and/or protein or expression patterns for DNA encoding the one or more biomarker, RNA transcript, protein, and/or a combination thereof of the one or more biomarkers disclosed herein in a biological sample obtained from a subject.

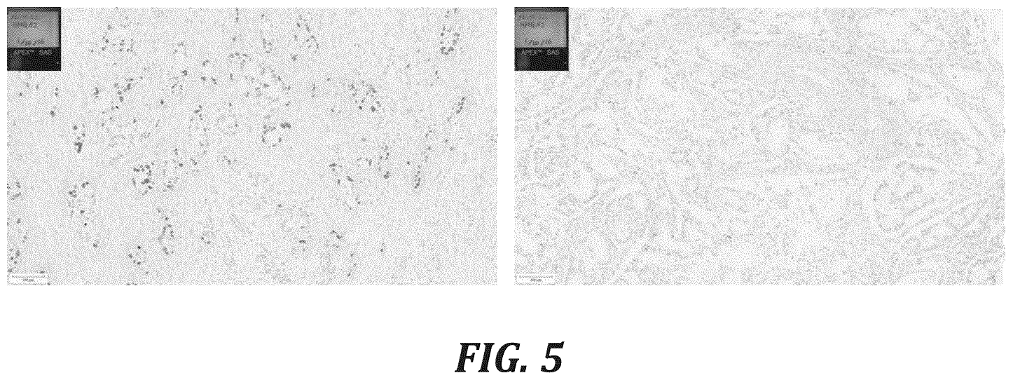

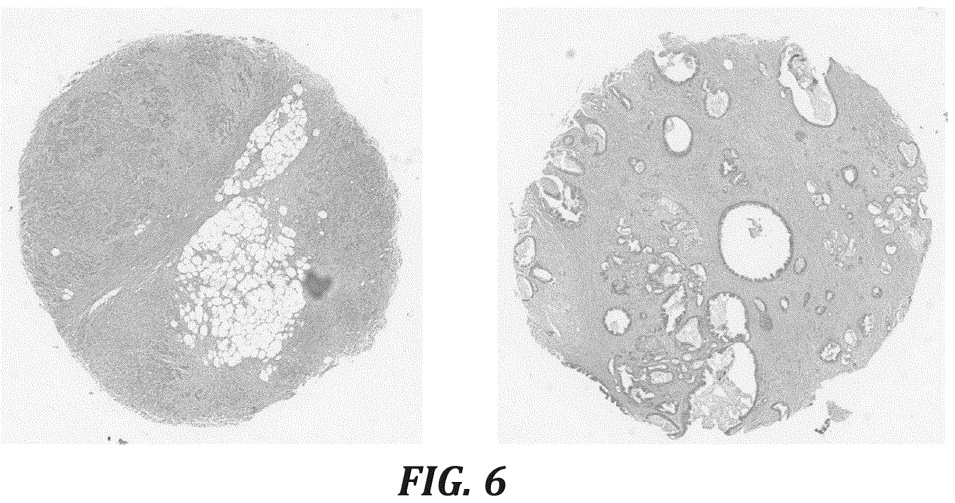

In one embodiment, the expression levels or levels of the at least one biomarker disclosed herein is determined using immunohistochemistry (IHC). In IHC, determining the level of a biomarker can be achieved by direct or indirect immunohistochemical detection of a biomarker using an appropriate antibody and detection reagents as known in the art. In both direct and indirect detection methods, the tissue/biological sample is treated to rupture the membranes, usually by using a kind of detergent, such as Triton X-100. Some antigens (i.e., the biomarker) also need an additional step for unmasking, resulting in better detection results. In direct detection, a labeled antibody (e.g., fluorescein isothiocyanate (FITC) conjugated antiserum) is reacted directly with the antigen/biomarker in tissue sections. In indirect detection, an unlabeled primary antibody is reacted with the antigen (i.e., the biomarker), and a secondary antibody is reacted with the primary antibody. The secondary antibody can be a labeled antibody, which is labeled, for example, with a fluorescent dye or an enzyme. A biotinylated secondary antibody can also be used. The biotinylated secondary antibody is detected with an enzymatic avidin or streptavidin conjugate, for example streptavidin-horseradish peroxidase. This exemplary conjugate can be reacted with 3,3′-Diaminobenzidine (DAB) to produce a brown staining wherever the primary and secondary antibodies have detected the biomarker. An exemplary detection of HMGA2 using IHC shows immunohistochemistry (IHC) for HMGA2^{high} and HMGA2^{low} in formalin fixed paraffin embedded (FFPE) human PDA tissue (FIG. 5).

Immunocytochemistry-based methods incorporating appropriate antibodies to the biomarkers and detection reagents are also known in the art. Similar to IHC, immunocytochemical methods determine the level of a biomarker using antibodies which can specifically bind to the biomarker of interest. Primary antibodies or antisera can be used for detection. A direct method can be used which incorporates a detectable tag (for example: a fluorescent molecule, gold particles, and the like) directly to the antibody that is then allowed to bind to the biomarker in a cell. Alternatively, an indirect method can be used. In one such method, the biomarker is bound by a primary antibody which is then amplified by use of a secondary antibody which binds to the primary antibody. The second antibody can incorporate a detectable tag, as described above. In another indirect method, a tertiary reagent could be used. A tertiary reagent could be bound to the secondary antibody could contain an enzymatic moiety. When an enzymatic substrate is applied, the enzymatic moiety converts the substrate into a detectable reaction product (e.g., a dye) in the same location as the biomarker. Some examples of substrates include AEC (3-Amino-9-EthylCarbazole), or DAB (3,3′-Diaminobenzidine). These reagents produce a detectable reaction product after exposure to the appropriate enzyme (e.g., horseradish peroxidase conjugated to an antibody reagent). Alternatively, the secondary antibody may be covalently linked to a fluorophore which is detected in a fluorescence or confocal microscope.

A number of proteomic techniques which can detect biomarkers are known in the art. Such techniques include, but are not limited to: Western blot analysis, enzyme linked immunosorbent assay (ELISA) or mass spectrometry. Matrix-assisted laser desorption/ionization (MALDI) is a mass spectrometry method for detecting biomarkers. In proteomics, MALDI can be used for the identification of proteins isolated through gel electrophoresis: SDS-PAGE, size exclusion chromatography, and two-dimensional gel electrophoresis.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Various protocols for conducting RIA to measure the levels of polypeptides in cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. Detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Different antibodies can be evaluated, and optimization procedures carried out (for example antibody dilutions and antigen retrieval methodologies), scoring systems and cut-off points of significance utilized, and various methods of data analysis can be employed according to this disclosure.

Determination of a biomarker can also be undertaken at the DNA level. Detection of DNA can be achieved using methods known in the art, such as Southern blot analysis, microarrays or other techniques known in the art. A Southern blot combines agarose gel electrophoresis for size separation of DNA with methods to transfer the size-separated DNA to a filter membrane for probe hybridization. As with a northern blot, the probe can be labeled so that it can be detected, usually by incorporating radioactivity or tagging the probe with a fluorescent or chromogenic dye. The hybridized probe, and by correlation the biomarker, can be visualized on X-ray film by autoradiography in the case of a radioactive or fluorescent probe, or by development of color on the membrane if a chromogenic detection method is used.

In accordance with one aspect, the differential expression patterns of the one or more biomarkers disclosed herein can be determined by measuring the levels of RNA transcripts of these genes, or genes whose expression is modulated by the genes, in the biological sample obtained from the subject. Suitable methods for this purpose include, but are not limited to, RT-PCR, Northern Blot, in situ hybridization, Southern Blot, slot-blotting, nuclease protection assay and oligonucleotide arrays.

Detection of RNA can be achieved using methods known in the art, such as northern blot analysis, microarrays, exon arrays or other transcriptome-based techniques. A northern blot is a method routinely used in molecular biology to check for the presence of an RNA sequence in an RNA sample. Northern blotting combines agarose gel electrophoresis for size separation of RNA with methods to transfer the size-separated RNA to a filter membrane for probe hybridization. The probe can be labeled so that it can be detected, usually by incorporating radioactivity or tagging the probe with a fluorescent or chromogenic dye. To ensure the specificity of the binding of the probe to the sample RNA, common hybridization methods block the membrane surface to reduce non-specific binding of the probe. Hybridized probe, and by correlation the biomarker, can be visualized on X-ray film by autoradiography in the case of a radioactive or fluorescent probe, or by development of color on the membrane if a chromogenic detection method is used.

In certain aspects, RNA isolated from the biological sample obtained from the subject can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes for the one or more biomarkers disclosed herein can be prepared from the gene sequence or obtained through commercial sources, such as Affymetrix. In certain embodiments the gene sequence is identical or complementary to at least 8 contiguous nucleotides of the coding sequence.

Sequences suitable for making probes/primers for the detection of their corresponding biomarkers include those that are identical or complementary to all or part of the SEQ ID NOs: 1-168, 170, and 172 described herein.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In another embodiment, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one embodiment, quantitative RT-PCR (such as TaqMan™, ABI) is used for detecting and comparing the levels of RNA transcripts in biological samples. Quantitative RT-PCR involves reverse transcription (RT) of RNA to cDNA followed by relative quantitative PCR (RT-PCR).

Yet another method for detecting RNA involves microarray. A microarray consists of an array of thousands of microscopic spots of oligonucleotides containing a specific sequence that are used as probes to hybridize a cDNA or cRNA sample (called target) under high-stringency conditions. Probe-target hybridization can be detected and quantified by fluorescence-based detection of fluorophore-labeled targets to determine relative abundance of nucleic acid sequences in the target. In standard microarrays, the probes are attached to a solid surface by a covalent bond to a chemical matrix. The solid surface can be glass or a silicon chip, or microscopic beads. Microarrays can be used to detect DNA (as in comparative genomic hybridization) or detect RNA (most commonly as cDNA after reverse transcription) that may or may not be translated into proteins.

Other techniques, for example, transcriptome-based techniques may be used in evaluation of RNA, as would be known to those of skill in the art.

Another powerful tool to visualize target DNA sequence or messenger RNA (mRNA) transcripts in cultured cells, tissue sections or whole-mount preparations of a biomarker in cultured cells, tissue sections or whole-mount preparations is a method broadly known as in situ hybridization (ISH). ISH functions via the principles of nucleic acid thermodynamics whereby two complementary strands of nucleic acids readily anneal to each other under the proper conditions to form a duplex (RNA: RNA or DNA: DNA), known as a hybrid. Under energetically favorable conditions, strands of RNA and DNA can also anneal to form DNA: RNA hybrids. These phenomena have facilitated the development of techniques that use either DNA or RNA probes to bind to DNA or RNA targets within a biological sample.

In an exemplary embodiment, the level of one or more biomarkers disclosed herein can be determined using in situ hybridization (ISH) technology. The method comprises using a probe comprising a labeled complementary nucleotide strand of deoxyribonucleic acid (DNA), ribonucleic acid (RNA), or a modified nucleic acids strand, to hybridize to, and identify, a specific DNA or RNA sequence of the one or more biomarker in a biological sample, a portion or section of tissue (in situ), in cells, and/or in circulating tumor cells.

In an embodiment, RNA-ISH comprises hybridizing the probe to an RNA, a portion of RNA, and/or a specific sequence of RNA of the one or more biomarkers disclosed herein. The RNA can be mRNA, Inc RNA, miRNA, and/or native RNA. RNA-ISH can measure and identify RNAs within tissue sections, cells, whole mounts, and circulating tumor cells.

The probe can be labeled with a fluorophore, radiolabel, or hapten-label. Suitable methods for detecting hybridization of the probe to its target are dependent on the label of the probe and can be selected accordingly by a person skilled in the art. Exemplary methods include but are not limited to analysis with a light or electron microscope. The RNA-ISH methods can also comprise whole mount in situ hybridization, double detection of RNAs (for more than one biomarker) and RNA plus protein, and/or fluorescent in situ hybridization to detect chromosomal sequences.

The nucleotide sequence of the probe can be complementary for the hmga2 gene or hmga2 RNA.

The nucleotide sequence of the probe can comprise a natural nucleotide sequence, a synthetic nucleotide sequence, and/or a nucleotide sequence which comprises one or more natural or synthetic modification. The nucleotide sequence of exemplary probes for the detection of HMGA2 and GATA6, using RNA-ISH are listed in Table 2. The probes disclosed herein can be optionally labeled with a fluorophore.

Despite their widespread use, conventional ISH approaches are still limited by technical considerations. For instance, it can be challenging to visualize hybridization events in complicated environments such as tissue samples due to inefficiencies in probe penetration and light collection.

Accordingly, several approaches have been developed to amplify the intensity of quantitative ISH signals. These strategies include the targeted deposition of detectable reactive molecules around the site of probe hybridization, the targeted assembly of ‘branched’ structures composed of DNA or locked nucleic acid (LNA) molecules, the programmed in situ growth of detectable con-catemers by enzymatic rolling circle amplification (RCA) or hybridization chain reaction (HCR), and the assembly of topologically catenated DNA structures using serial rounds of chemical ligation.

In an embodiment, the sensitivity of the detection of the one or more biomarkers by ISH can be further amplified by signal amplification exchange reaction (SABER). In an embodiment, the SABER methodology endows oligo-based ISH probes with long, single-stranded DNA concatemers that serve as targets for sensitive fluorescent detection. This technology leverages a molecular strategy to program the autonomous synthesis of ssDNA in vitro termed ‘primer ex-change reaction’ (PER), which enables the growth of long ss-DNA concatemers from a short (9 nt) DNA primer sequence. By designing a single PER hairpin sequence to act as a catalytic template, identical sequence domains can be repeatedly appended to nascent single-stranded primer sequences with a strand displacing polymerase. SABER harnesses the programmability of PER to enhance the functionality of oligo-based FISH probes such as single-molecule RNA FISH probe pools and highly complex ‘Oligopaint’ probe sets. Briefly, DNA and RNA FISH probes are first chemically synthesized with primer sequences on their 3′ ends and then concatemer-ized using PER in vitro. Extended probe sequences are then hybridized in situ and act as scaffolds to which multiple fluorescent strands can bind. SABER effectively amplifies the signal of probes targeting nucleic acids in fixed cells and tissues and can be deployed against multiple biomarkers/targets simultaneously and detects mRNAs with high efficiency.

Also disclosed herein is a kit, comprising reagents for detecting or measuring the levels of one or more biomarkers disclosed herein. In an embodiment, the disclosure comprises a kit, wherein the kit comprises a molecule that binds to a PDA cell or a component of a PDA cell. In an embodiment, the molecule is a probe comprising at least one of the nucleotide sequences as set forth in Table 2, or a portion thereof. In an embodiment, the at least one nucleotide sequence is optionally labeled with a fluorophore.

### EXAMPLES

### Example 1

### Identification of HMGA2^{high} PDA as a Biomarker for Treatment

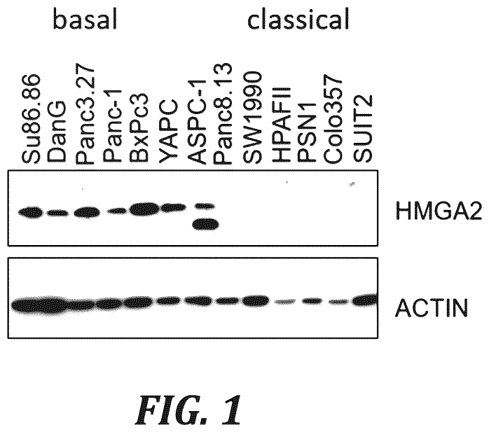

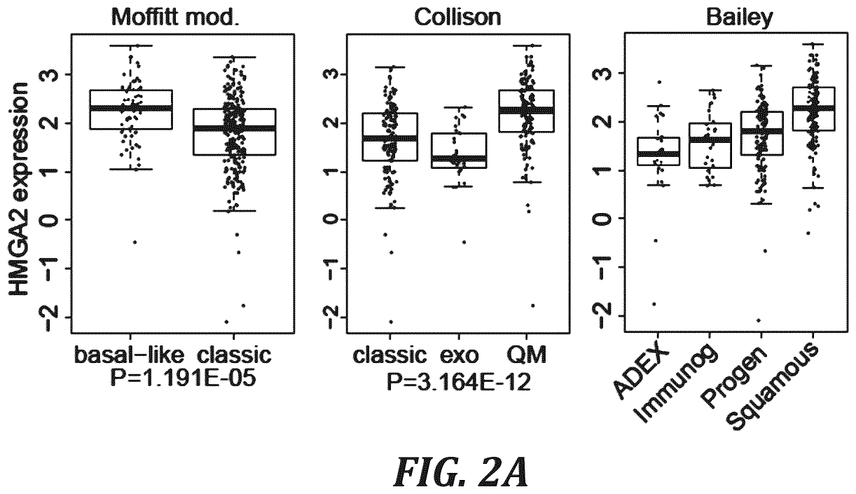

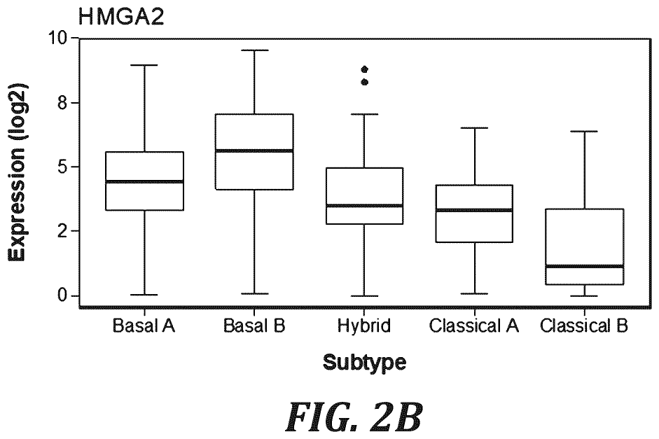

HMGA2 and ACTIN in basal PDA (red), compared with classical PDA (black) cell lines is shown in a Western blot in FIG. 1. HMGA2 is expressed at higher levels in basal PDA, as shown in FIG. 2B.

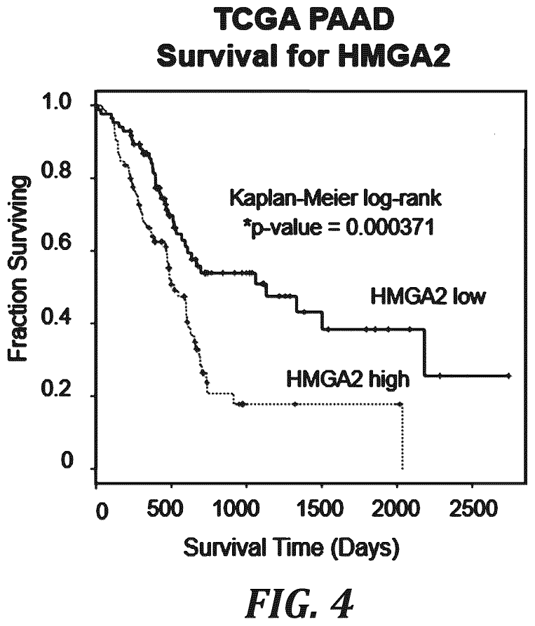

Moreover, analyses of proteomic data from TCGA (The Cancer Genome Atlas) PDA samples indicated higher levels of HMGA2 correlated with a worse overall survival (FIG. 4).

To assess whether HMGA2^{high} PDA correlated with treatment response, the Chan-Seng-Yue dataset (Nat. Genet. 52 (2): 231-240, 2020) of subjects was analyzed. The study cohort included 206 patients with resectable (stage I/II) PDA, and 111 patients with advanced (stage III/IV; total 317 patients) PDA. Biospecimens underwent laser capture microscopy (LCM) to improve tumor cellularity for genomic analyses and were subjected to whole-genome sequencing (WGS) (n=330 samples from 314 patients) and RNA-seq (n=248 from 242 patients; 239 patients had paired WGS).

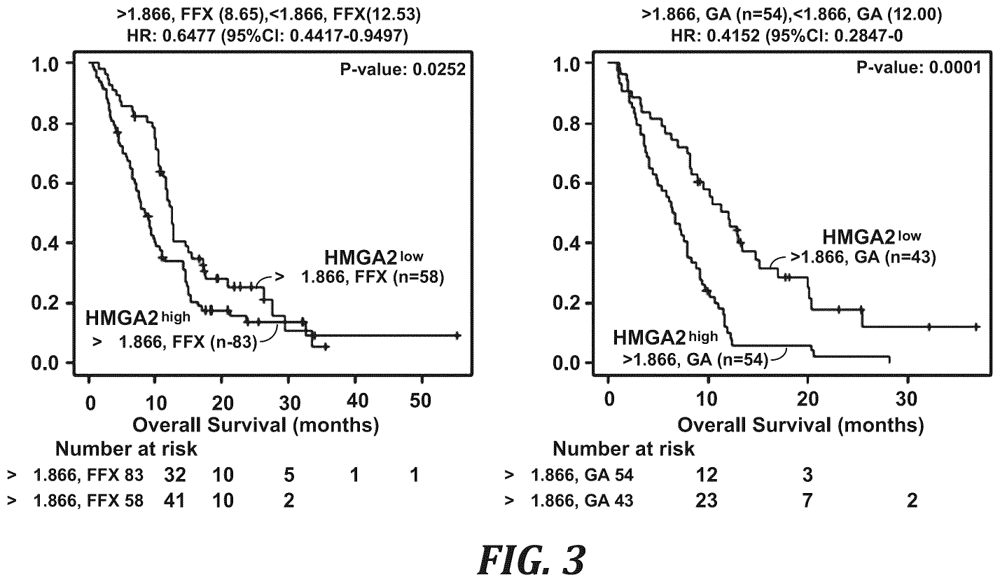

The HMGA2 high state/basal subtype predicted efficacy from GA. Patients with advanced disease and high versus low levels of HMGA2 had shorter overall survival (6.58 months for HMGA2^{high} vs 12 months for HMGA2^{low}, HR: 0.4152, p value: 0.0001) when treated with GA as first line therapy (FIG. 3). Thus, the HMGA2 as a predictor for GA sensitivity was identified.

### Example 2

### HMGA2 as a Novel Biomarker in PDA

Patients with PDA that express higher levels of HMGA2 demonstrate reduced overall survival when treated with GA. Primary and paired metastatic lesions from 10 patients through the Center for Accelerated Translation in Pancreas Cancer (CATPAC) Rapid Autopsy Program (RAP) were harvested (Institutional Review Board (IRB) protocol 2296.00; PI Hingorani). This allows for the rapid acquisition and preservation of malignant and benign tissues from recently deceased patients. These specimen types further the studies of tumor evolution, differences between primary tumor and metastases, and understanding the effects of therapy response and resistance.

All samples undergo targeted Next Generation Sequencing (NGS) and bulk RNA-seq, and the de-identified raw data were deposited into OncoScape. The molecular and clinical data was aggregated to improve precision oncology care.

The assay is a 648-gene DNA panel to sequence both tumor tissue and matched normal specimens (peripheral blood or saliva), along with full transcriptome RNA sequencing of the tumor specimen. The PDA assigner algorithm and RNA-seq data is used to characterize the primary and metastatic tumors as basal or classical subtype.

HMGA2 expression levels from RNA-seq data of human PDA samples were classified as belonging to either basal/QM/squamous (red) or classical/progenitor (black) subtypes. 236 subjects had advanced stage pancreas cancer, 139 patients received FFX, and 97 received first line therapy GA. Biospecimens underwent laser capture microscopy to improve tumor cellularity for genomic analyses and were subjected to whole-genome sequencing and RNA-seq. Sec FIG. 2A.

Gene set enrichment analysis is performed using Gene Set Enrichment Analysis (GSEA) (v.3.0) and gene signatures extracted from Bailey, et al. (Nature 531:47-52, 2016) to calculate an FDR-controlled q-val based on the hypergeometric distribution. The gene set enrichment analysis (GSEA) is then used to determine whether HMGA2 is enriched in basal versus classical tumors in subjects. Clinical data is correlated, and the overall survival in days from the time of PDA diagnosis until date of death is calculated.

By using the log-rank test for univariate association with survival, the basal gene signature, or high HMGA2 levels, correlating with survival in patients treated with GA or FFX as first line therapy is identified.

The LENS database of clinically annotated patients is queried to validate HMGA2 as a biomarker for GA response. This allows supplementation of internal data with data from other institutions, to increase patient number.

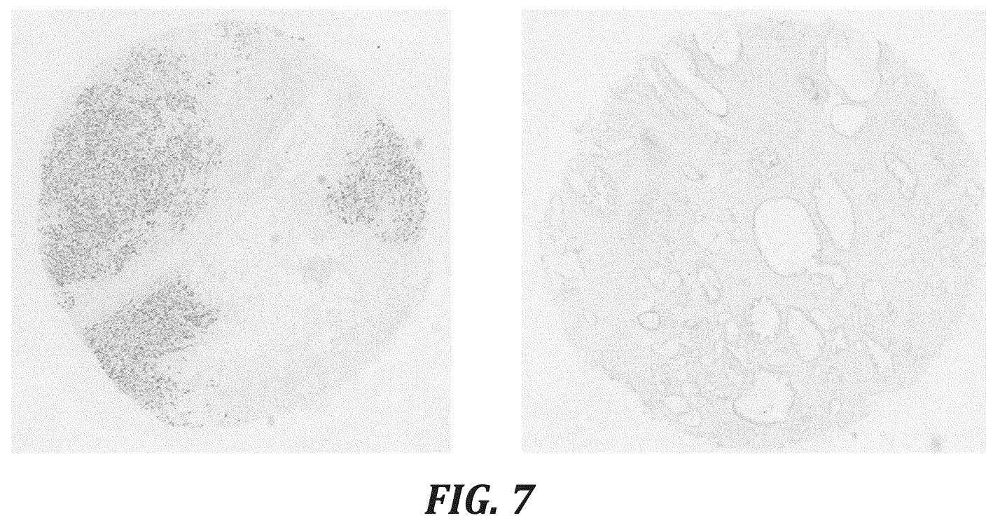

Paired formalin fixed paraffin embedded (FFPE) resected human PDA tissue samples for HMGA2 by immunohistochemistry (IHC) are stained. See FIG. 5 and FIG. 7. This permits identification of basal/HMGA2^{high} and classical/HMGA2^{low} tumors and determination of whether HMGA2 protein levels correlate with HMGA2 transcripts as measured by RNA-seq. These data demonstrate that HMGA2 levels by IHC can be used as a clinical test to inform patient treatment.

Additionally, HMGA2 status is co-analyzed with transcriptional subtype, treatment response, and survival data, which determines whether high expression of HMGA2 correlates poor survival with GA treatment. Similar to the Chan-Seng-Yue dataset (Nat. Genet. 52 (2): 231-240, 2020), HMGA2 is expected to be enriched in the basal subtype, and high HMGA2 levels are expected to correlate with poor survival in patients treated with GA in both the RAP samples and the Tempus database.

Protein levels of HMGA2 by IHC are expected to closely match RNA levels detected by RNA-seq.

### Example 3

### HMGA2 to Predict Nab-Paclitaxel Plus Gemcitabine Sensitivity In Vivo

Two successful PDA patient-derived xenografts (PDXs) from resected patient tumors have been directly implanted into immunocompromised mice. In addition, established PDA PDXs from the National Cancer Institute Patient-Derived Model Repository (PDMR) (n=8) and JAX (n=3) have been revived and expanded for drug trials. Tissue samples from a RAP patient are collected and organoids from both primary and metastatic lesions generated. This demonstrates functionality of the pipeline expanded to include the generation of PDXs. HMGA2^{high} PDA PDXs are expected to be more resistant to GA; HMGA2 status will not determine sensitivity to FFX.

Tumor chunks (200 mg) from 2 primary tumor samples, 3 liver metastases, and 3 lung metastases, from the same subject, are implanted into immunocompromised mice. Performing these studies in vivo in a curated and annotated set of PDXs from patients with a known response to a given chemotherapy regimen allows for guiding clinical studies. The response of these PDXs to GA or FFX is determined using established protocols. PDXs in immunocompromised mice (10 mice/cohort) are established.

Mice with demonstrable primary tumors (200-300 mm³) are selected and randomly allocated into the following arms: (1) Control: saline i.v. (D1, D8, D15); (2) Gemcitabine: 200 mg kg⁻¹, i.v. 3 weeks on 1 week off (D1, D8, D15); (3) nab-paclitaxel: 40.5 mg kg⁻¹, i.v, 3 weeks on 1 week off (D1, D8, D15); (4) nab-paclitaxel plus Gemcitabine: at the above-mentioned doses i.v. nab-paclitaxel (D1, D8, D15) and Gemcitabine (D2, D9, D16); (5) FOLFIRINOX: 66.5 mg kg⁻¹ i.v. folinic acid, 14 mg kg⁻¹ i.v. oxaliplatin, 50 mg kg 1 (pellet) fluorouracil, and 30 mg kg⁻¹ i.v. irinotecan all drugs dosed once every 2 weeks).

Drug doses are selected based on previous studies. Tumor size is monitored by caliper measurements 3×/week to generate a growth curve for a period of 28 days or until tumor size necessitates euthanasia.

A subset of tumors are used to characterize acute histopathologic changes resulting from GA or FFX treatment. HMGA2 expression is confirmed by comparing levels of HMGA2 in flash-frozen tumor chunks by western blot (WB) and IHC of HMGA2 in FFPE tissue sections. Mitotic index and apoptosis in tumor tissue sections harvested from cohorts of mice at 10 days following drug treatment are quantified.

Patient-derived basal/HMGA2^{high} and classical/HMGA2^{low} organoids are generated. Organoids are implanted orthotopically directly into the pancreas of immunocompromised mice (10 mice/cohort) and tumor growth is monitored weekly using a Vevo 770 high-frequency ultrasound system.

Mice with demonstrable primary tumors (200-300 mm³) are selected and randomly allocated into treatment and control arms. This approach assesses the efficacy of GA or FFX sensitivity in tumors growing within the pancreas.

Additionally, in vitro cytotoxicity of organoids is evaluated to determine correlation with in vivo efficacy and clinical response. Organoids are dissociated into a single cell suspension and 500 live cells per well are plated in triplicate in a 384-well plate. Organoids are treated with vehicle control or drugs the following day with a minimum of 9 doses. Five days after treatment, cell viability is assessed by measuring intracellular ATP levels using CellTiter-Glo® 3D Reagent.

Already-established PDA PDX models from NCI PDMR and JAX can be used upon engrafting failure of PDXs from rapid autopsy. Additionally, a new subrenal capsule implantation method to improve take rates for PDA PDXs can be used.

These experiments provide vital preclinical data for the development of a novel biomarker-driven clinical treatment of human PDA, where HMGA2 expression is used to select patients for GA or FFX treatment.

### Example 4

HMGA2 to Predict Efficacy of Treatment with Nab-Paclitaxel Plus Gemcitabine Vs FOLFIRINOX for Patients with Advanced PDA

The role of HMGA2 expression prospectively in subjects undergoing treatment with GA or FFX is tested. Approximately 300 new patients per year are tested, through twice weekly multidisciplinary pancreatic cancer clinics, and multiple individual provider clinics each week. Approximately 50% of patients have metastatic PDA and another 25% have locally advanced unresectable PDA.

All patients undergo tumor biopsy at baseline, preferentially from metastatic sites, and all undergo molecular profiling through the Next Generation Sequencing (NGS) PFS and transcriptomic platform described above. Approximately 50% of patients undergo 1st line treatment with GA and 50% of patients undergo 1st line treatment with FFX. Upon progression from 1st line therapy, 2^{nd} line treatment options include GA (for patients treated with 1st line FFX) or 5-FU based treatments (for patients treated with 1st line GA).

Patients with pancreas cancers with HMGA2^{high} levels treated with GA will likely have lower response rates, lower progression-free survival (PFS), and decreased overall survival (OS) compared to patients with cancers with HMGA2^{low} levels treated with GA.

The status of HMGA2 by IHC and comprehensive molecular genomic and transcriptomic profiles are performed on the collected samples. All patients consent to allow investigators access to their EPIC electronic medical records, and to allow survival follow-up. Demographics, histopathological data, treatment type, best response to treatment by RECIST1.1 criteria, progression-free survival (time from start of treatment to disease progression, death, or last follow-up), overall survival (time from start of treatment to death of any cause or last follow-up) are collected. Both 1st line and 2nd line treatment data are collected.

Tumor response and disease status is determined by imaging with i.v. contrast CT scans chest/abdomen/pelvis or MRI imaging every 6-8 weeks, per standard of care. CA19-9 or CEA markers (when CA19-9 is normal) are collected every 4 weeks. HMGA2 expression levels are correlated with response, progression free survival (PFS) and overall survival (OS) for patients with locally advanced and metastatic PDA, and patients treated with GA and with FFX in 1st and 2nd line setting.

When subjects choose to undergo treatment in local communities, the local treating physician is contacted to obtain the status of the disease (progressing or not) and survival, and the best response to treatment per RECIST (response evaluation criteria in solid tumors) criteria is ascertained with imaging records.

Prospective validation of the role of HMGA2 to predict response to treatment and prognostic outcomes informs best treatment decisions to be made for patients with advanced PDA.

IRB approval was sought to derive organoids from needle biopsy specimens. HMGA2 protein levels were evaluated by Western blot (FIG. 1), and RNA levels were determined by qRT-PCR and compared to RNA-seq data. In vitro cytotoxicity assays were then carried out as previously described, and the data correlated with overall survival and progression-free survival to determine organoid testing prediction for sensitivity to GA and FFX. This sensitivity data is used to inform treatment decisions.

The subjects characterized as HMGA2^{high} showed significantly shorter survival when GA treated. See FIG. 3 (right panel). Therefore, a characterization of HMGA2^{high} informs the treating physician to prescribe a FOLFIRINOX treatment regimen for first line therapy for subjects with PDA identified as HMGA2^{high}.

### Example 5

### Characterizing HMGA2 by RNA-ISH

An innovative branched nucleic acid RNA-in situ hybridization (ISH) technology called ViewRNA™ permits ready detection of RNA transcripts in archived FFPE sections. This assay is adaptable for clinical use. RNA FISH is a type of RNA-ISH.

All procedures were performed at room temperature, except where otherwise specified.

### RNA SABER-FISH Protocol

Primer preparation: Primers were designed using PaintSHOP (10.1038/s41592-021-01187-3) with p27 appended at 3′ end and ordered as oPools™ oligo pools from IDT. Polymerase extension (PE) reactions to add p27 hairpin were performed in 1×PBS with 1 μM CleanG hairpin, 6 mM dNTPs (A,C,T only), 100 mM MgSO₄, and 8 U/μL Bst LF polymerase (IDT). 10 μL of 5 μM hairpin was added, then incubated for 15 min at 37° C. 10 μL of 10 μM target probe was added, then extension was performed at 37° C. for two hours, followed by heat inactivation at 80° C. for 20 minutes. Following confirmation that reaction was successful by running on 5% Criterion TBE/Urea gel (Bio-Rad) at 180 V for 35 min, 24 μL 1 M PER per slide probed was vacuum dried.

Sample preparation: FFPE slides were baked at 55° C. overnight, then de-paraffinized with 100% xylenes twice (3 min), 100% EtOH twice (2 min), 95% EtOH (2 min), 80% EtOH (2 min), dH₂O (2 min). Samples were then treated with RNAscope™ Hydrogen Peroxide solution for ten minutes, rinsed twice in dH₂O, and then boiled in 10 mM citrate buffer (pH 6) for 20 min at 95° C. Following two rinses in dH₂O and two rinses in 100% EtOH, hydrophobic barrier was drawn with pen and samples were treated with a mild protease treatment (RNAscope™ Protease Plus) at 40° C. for 30 min in humidified chamber. Samples were kept in dH₂O until ready to use (no longer than 2 hours).

Primary hybridization: Slides were washed in 1×PBS for 1 min, followed by 1×PBS (0.5% Triton X-100) for ten minutes, then 1×PBS (0.1% Tween) for 2 min. After a five minute 0.1N HCl wash, slides were washed twice in 2×SSC (0.1% Tween) for 1 minute, then 2×SSC (0.1% Tween+50% Formamide) for 2 min, then 2×SSC (0.1% Tween+50% Formamide) for 20 min at 60° C. Primary probes were resuspended in primary hybridization solution (2×SSC, 0.1% Tween, 40% Formamide, 10% dextran sulfate) to a concentration of 1 μM. 40 μL of solution was applied to each slide, then coverslips were applied and sealed with rubber cement. Samples were denatured at 60° C. for 3 min, then incubated overnight at 37° C. in a humidified chamber.

Secondary hybridization: Rubber cement and coverslips were removed, then slides were rinsed with pre-warmed 60° C. 2×SSC (0.1% Tween) twice. Following four 5-min washes with pre-warmed 60° C. 2×SSC (0.1% Tween) and two 2-min washes with room temperature 2×SSC (0.1% Tween), samples were rinsed in PBS. 40 μL of secondary hybridization solution (1:250 100 μM secondary probe in PBS) was added to each sample, then sealed with cover slips and rubber cement. Samples were incubated in humidified chamber at 37° C. for 1 hour, rinsed with PBS (0.1% Tween), followed by two 5-min washes with pre-warmed 37° C. PBS (0.1% Tween), then one 5-min wash with pre-warmed 37° C. PBS. Slides were mounted with 8 μL an antifade slide cover mountant (SlowFade™)+DAPI and coverslips were applied.

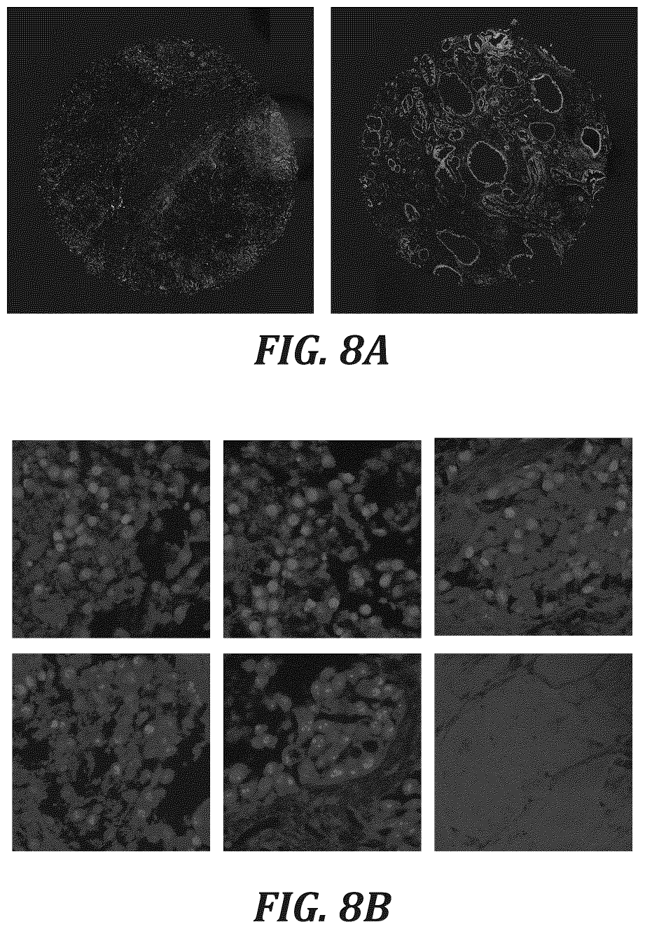

FIG. 8B shows the in-situ hybridization (ISH) for HMGA2 levels in formalin fixed paraffin embedded (FFPE) human PDA tissue. FIG. 8A shows HMGA2^{high} (left) and HMGA2^{low} (right) expression in formalin fixed paraffin embedded (FFPE) human PDA tissue, using in situ hybridization (RNA ISH). HMGA2^{high} appears red. HMGA2^{low} appears green. FIG. 8B shows GATA6 (upper left panel), GATA6 (upper center panel) expression as determined by RNA ISH in subcutaneous tumors of human PDA cell lines along with HMGA2 detection (FIG. 8B, upper right panel). FIG. 8B lower panel shows positive controls detecting nucleolar ITS transcript (FIG. 8B, lower left and center panel) and a negative control (FIG. 8B, lower right panel).

### Example 6

### HMGA2 and GATA6 Status Predicts Survival

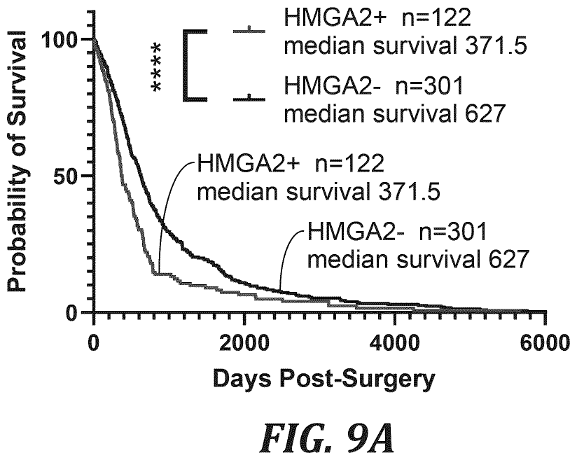

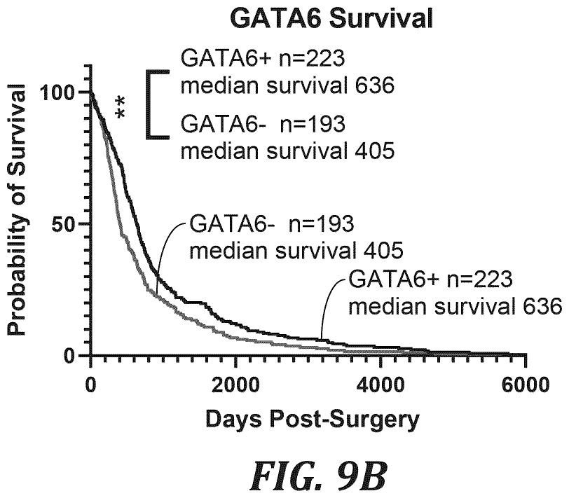

Expression levels of HMGA2 and GATA6 were determined separately in Tissue microarray comprising human PDA tumors from over 800 patients, along with matched (normal pancreatic tissue) controls and correlated to clinical data (stage at diagnosis, treatment history). These samples were stained and scored for HMGA2 (FIG. 9A) and GATA6.

Concentrations of antibodies used are shown in Table 1 below:

TABLE 1
Antibody concentrations

| | | | | Conc/ | Opal |
|---|---|---|---|---|---|
| Position | 1° Ab | Host/Clone | Vendor/Cat # | Dil | dye |
| 1 | FAP | Rb/poly | Abcam ab53066 | 1:250 | 480 |
| 2 | HMGA2 | Rb/poly | CST 5269 | 1:150 | 520 |
| 3 | CK17 | Rb/EP1623 | Abcam ab109725 | 1:1000 | 570 |
| 4 | CD45 | Rb/EP322Y | Abcam ab40763 | 1:1500 | 620 |
| 5 | CK5 | Ms/XM26 | Leica | 1:1000 | 690 |
| | | | NCL-L-CK5 | | |
| 6 | GATA-6 | Rb/D61E4 | CST 5851S | 1:1000 | 780 |
| Nuclear | DAPI | | | 1 μg/ml | |
| stain | | | | | |

Patients with a HMGA2^{high} and GATA6^{low} status showed low median survival, 371.5 days, and 405 days, respectively, post-surgery, while patients with HMGA2^{low} and GATA6^{high} show a higher median survival, 627 days and 636 days, respectively, post-surgery (FIG. 9B).

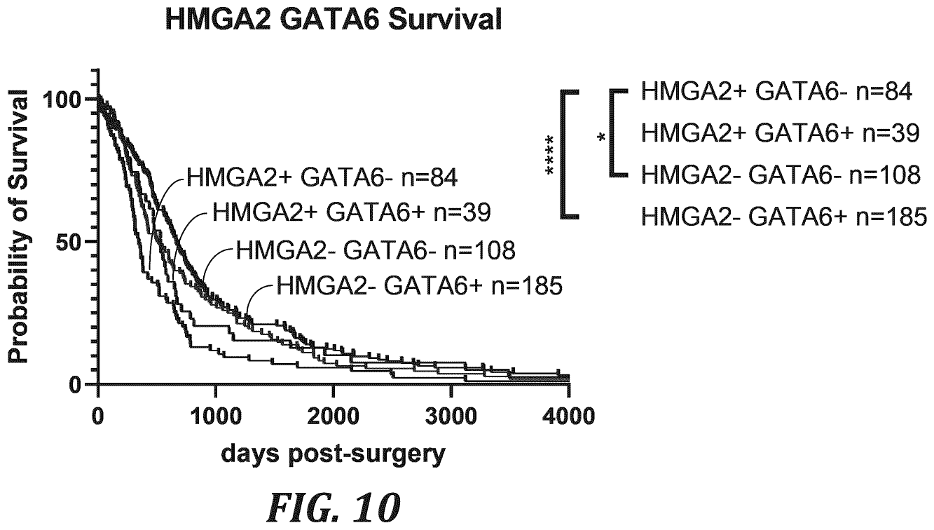

Overall probability of median survival post-surgery based on expression levels of both HMGA2 and GATA6 identified four separate groupings based on HMGA2 and GATA6 expression levels with HMGA2^{high}/GATA^{low} showing the poorest overall median survival and HMGA2^{low}/GATA6^{high} showing the best overall median survival (FIG. 10).

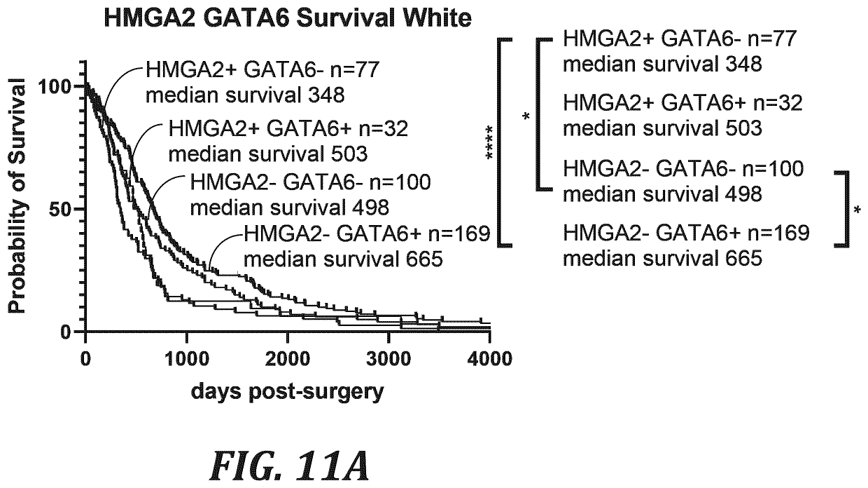

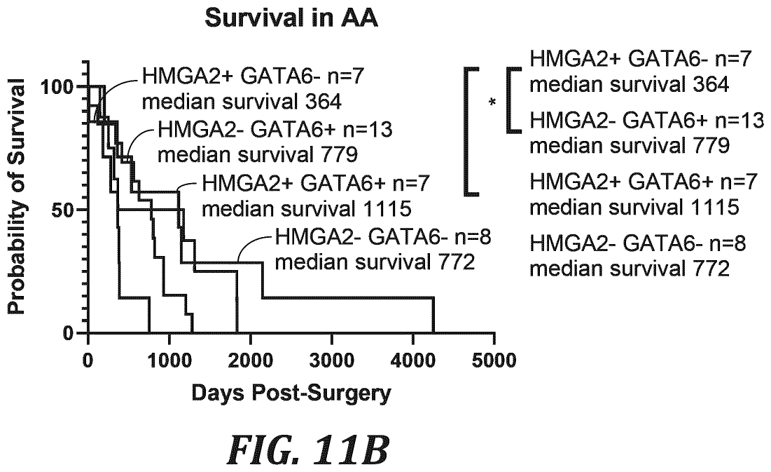

Further, the use of both HMGA2 and GATA6 as biomarkers allowed for accurate prediction of response to overall median survival in Caucasian (FIG. 11A) and black populations (FIG. 11B).

### Example 7

### HMGA2 and GATA6 Status Predicts Response to First-Line Therapy

HMGA2 expression in metastatic and resectable patient samples from the COMPASS trial is higher in the basal subtype. Samples were purified by laser capture microdissection before RNA sequencing. FIG. 3 shows overall median survival for 236 advanced stage patients, out of which 139 patients received FFX, and 97 patients received GA first line therapy as determined by HMGA2 expression and response to chemotherapy. The HMGA2^{high} state/basal subtype predicted efficacy from GA. Patients with advanced disease and high versus low levels of HMGA2 had shorter overall survival (6.58 months for HMGA2^{high} vs 12 months for HMGA2^{low}, HR: 0.4152, p value: 0.0001) when treated with GA as first line therapy.

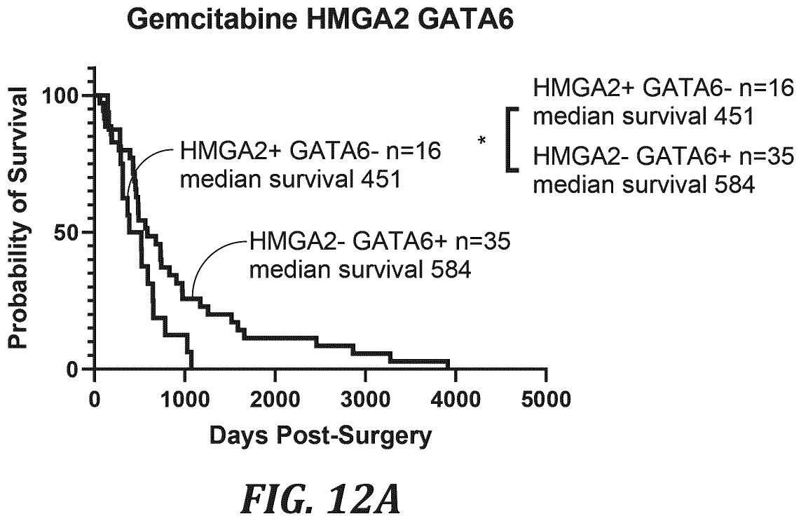

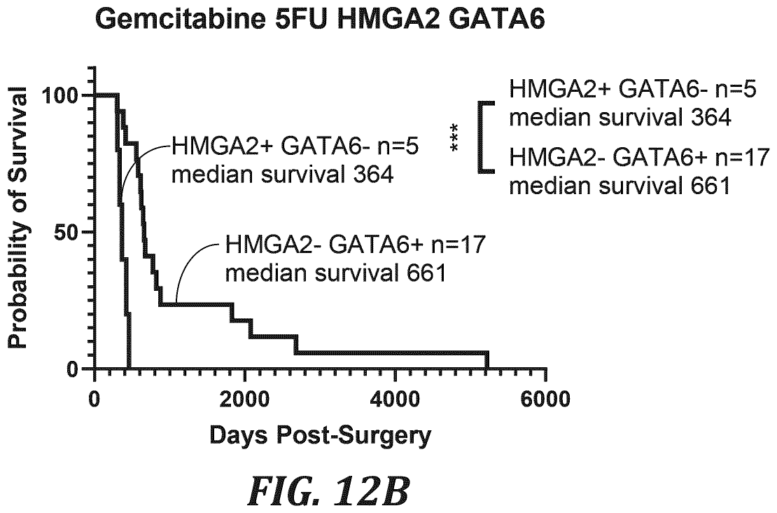

HMGA2/GATA6 predict prognosis to Gemcitabine treatment. HMGA2^{high}/GATA6^{low} expressing tumors show an overall low median survival when treated with Gemcitabine as first-line therapy as compared to HMGA2^{low}/GATA6^{high} tumors treated with Gemcitabine as first-line therapy (FIG. 12A). HMGA2^{high}/GATA6^{low} expressing tumors show an overall low median survival when treated with Gemcitabine/5 FU as first-line therapy as compared to HMGA2^{low}/GATA6^{high} tumors treated with Gemcitabine/5 FU as first-line therapy (FIG. 12B).

TABLE 2
Probes and Sequences

| PROBE | SEQ ID NO: | SEQUENCE |
|---|---|---|
| HMGA2_human | 1 | GAGAGGTATGTGGCCTTTGAAACTACCTCCCTGAATTTACATCATCATACATCATCAT |
| HMGA2_human | 2 | AAGCAAAGGAGGATGGGGAGACTCCGCCGGTTTACATCATCATACATCATCAT |
| HMGA2_human | 3 | GCAGCTGGAAGAGAGATGGTTTTGAGTTTCATTTGGCTTTACATCATCATACATCATCAT |
| HMGA2_human | 4 | GATAGGGTCGGGCACGGAGCACAGGCAGAGTTTACATCATCATACATCATCAT |
| HMGA2_human | 5 | GGCTTGTTGCATCTCACCTACAAAGCTTCAAGTTTCTTTTACATCATCATACATCATCAT |
| HMGA2_human | 6 | TGGCCAATGAGGTTTCTAAAAGGCCCATCCAAAGATATTTACATCATCATACATCATCAT |
| HMGA2_human | 7 | CTGGAAGTTTTCTGAGTTCCTGCCCCAAGATTCAAGTTTACATCATCATACATCATCAT |
| HMGA2_human | 8 | CTAGCTCCTGAGTCTTGCACCAAGCGCGCGTTTACATCATCATACATCATCAT |
| HMGA2_human | 9 | CAGAGTAGTGGGTGGCACCGCGCCTCCTAGTTTACATCATCATACATCATCAT |
| HMGA2_human | 10 | TGGGTCTTCCCCTGGGTCTCTTAGGAGAGGTTTACATCATCATACATCATCAT |
| HMGA2_human | 11 | GGAACAGGGAGAAAGTCAACTGCAGTTAAACGTCTTGTTTACATCATCATACATCATCAT |
| HMGA2_human | 12 | TCCTTTTGAAGTCTGGAGGTAGGTCTTTGCAAGTCAATTTACATCATCATACATCATCAT |
| HMGA2_human | 13 | ATCAGGAGTTGCGGGCACCGGGAGCACTTCTTTACATCATCATACATCATCAT |
| HMGA2_human | 14 | CGTCATGAGAAACTACCTCCTGGCCCAGTTGATAATTTTACATCATCATACATCATCAT |
| HMGA2_human | 15 | CCATTTCCTAGGTCTGCCTCTTGGCCGTTTTTTTACATCATCATACATCATCAT |
| HMGA2_human | 16 | AGGTTGTCCCTGGGCTGAAGTGGACGGCTGTTTACATCATCATACATCATCAT |
| HMGA2_human | 17 | TGATTCACAACAGTGAGCGATCTGCCAGTCTCATTTACATCATCATACATCATCAT |
| HMGA2_human | 18 | CTTTGAGGTCGCAGAGGCTCCTCTCGCGGGTTTACATCATCATACATCATCAT |
| HMGA2_human | 19 | CCATGTTTGGCCAACATGAGCAATTGCTTCCATTTTTTTACATCATCATACATCATCAT |
| HMGA2_human | 20 | GCTGCGACCAACAACAGCAAAGAACAGTCCTATAAAATTTACATCATCATACATCATCAT |
| HMGA2_human | 21 | AGGGAAGAGACTTGGAGTGAATTGTGTCCCTTGAAATTTTACATCATCATACATCATCAT |
| HMGA2_human | 22 | TGGGGATCACAGAGGCTGTTATGTTTATTGTGCAGAATTTACATCATCATACATCATCAT |
| HMGA2_human | 23 | GAGATGAGGTGATAGGGCTGGGGACGCCGGTTTACATCATCATACATCATCAT |
| HMGA2_human | 24 | GCTGAGGTAGAAATCGAACGTTGGCGCCCCTTTACATCATCATACATCATCAT |
| HMGA2_human | 25 | TGCAGTGTCTTCTCCCTTCAAAAGATCCAACTGCTTTTACATCATCATACATCATCAT |
| HMGA2_human | 26 | ACCTGGGACTGTGAAGGGATTACAAAGAAGGTGATTTTTACATCATCATACATCATCAT |
| HMGA2_human | 27 | CATTGTTAAGCTGTGTCCCCTGTGTTTACAGCAGTTTTTTACATCATCATACATCATCAT |
| HMGA2_human | 28 | ACCCGCCCACTCTTGCTTATCAGATCAACAAACTATTTTTACATCATCATACATCATCAT |
| HMGA2_human | 29 | TGCAGTGCAGTGATTGACTAAACCCAATTCGGTTTTTTTACATCATCATACATCATCAT |
| HMGA2_human | 30 | CGACGTCACAAGTGTGACACGTTTCTTGTTTGCATTTACATCATCATACATCATCAT |
| HMGA2_human | 31 | ACAGTGTTACACACCGCGTTCTTCCTATATGAATGCCTTTACATCATCATACATCATCAT |
| HMGA2_human | 32 | GGGAGTGGGTTGGGGTGGTATTTGAGGTGTACTTTACATCATCATACATCATCAT |
| HMGA2_human | 33 | GCTCCTCCCACCTCATAATTAGGTGAGTTCATCATGCTTTACATCATCATACATCATCAT |
| HMGA2_human | 34 | GCCGAAGAAAAGCACCTTGGTCAACCATCTTATGTCATTTACATCATCATACATCATCAT |
| HMGA2_human | 35 | AGTGGGGATATATTGCATCTCTGGCTAAAAGTGCAGTTTTACATCATCATACATCATCAT |
| GATA6_human | 36 | GTGCTCTCTCCCGCACCAGTCATCACCGGGTTTACATCATCATACATCATCAT |
| GATA6_human | 37 | CCGAATACTTGAGCTCGCTGTTCTCGGGATTGTTTACATCATCATACATCATCAT |
| GATA6_human | 38 | AAAGCAGACACGAGTGGAGTGAGGCCCGCGTTTACATCATCATACATCATCAT |
| GATA6_human | 39 | TCTGTCAGCGCAGTCGCCACTGTCTGGACCTTTACATCATCATACATCATCAT |
| GATA6_human | 40 | CGAGACTGACGCCTATGTAGAGCCCATCTTGACTTTACATCATCATACATCATCAT |
| GATA6_human | 41 | AATCCGGTCGCACGGAGGACGTGACTTCGGTTTACATCATCATACATCATCAT |
| GATA6_human | 42 | TCCATAGCAGGCAAGGCCCAGGTCCTAGTCTTTACATCATCATACATCATCAT |
| GATA6_human | 43 | CCATGAACGCACATGAAATCTGGATGTGGAAAAGGTTTTACATCATCATACATCATCAT |
| GATA6_human | 44 | TCCGACTGACTTCAGATCAGCCACACAATATGAACTTTTTACATCATCATACATCATCAT |
| GATA6_human | 45 | CCTGCCTGTGGGTTAGTCACACTATACAGACTTCATCTTTACATCATCATACATCATCAT |
| GATA6_human | 46 | GCCTTAAAGCATTTTGCAAACTTCACGTGCACTTGTTTTTACATCATCATACATCATCAT |
| GATA6_human | 47 | ACAAAACGGCTCCAAAAGGACTATGCTTTGAAAGGAATTTACATCATCATACATCATCAT |
| GATA6_human | 48 | GCACATGGAGAGTAGTAACTAACCCCATGTGTCAACTTTTACATCATCATACATCATCAT |
| GATA6_human | 49 | GCCCTGTTCGTGGGCCAGAATATATTTCATTCACAAATTTACATCATCATACATCATCAT |
| GATA6_human | 50 | AGCTTTGAGACTTCAAGGCACGTTGCAAAAGGAAAATTTACATCATCATACATCATCAT |
| GATA6_human | 51 | GGCCACTGGAATCATTTCTACCTATCTTCTGTTGGGGTTTACATCATCATACATCATCAT |
| GATA6_human | 52 | TCTTCTCTCACTCTTTCAGCAAGCCTCTTGGGAAAATTTACATCATCATACATCATCAT |
| GATA6_human | 53 | CTGGCTTCTGGAAGTGGAGCAGGGTCCGCCTTTACATCATCATACATCATCAT |
| GATA6_human | 54 | AAACAAACAGCGCTCGCCGAAGGGTGCCAGTTTACATCATCATACATCATCAT |
| GATA6_human | 55 | CTGGGCTCCTGATTGGACTCACCGAGCCCTTTTACATCATCATACATCATCAT |
| GATA6_human | 56 | CCTCTTACTGCTCTGCCGGAAAACTGCAGCTTTACATCATCATACATCATCAT |
| GATA6_human | 57 | GGGTCTGCGCCGCGCTGCTGGTGAATAAAATTTACATCATCATACATCATCAT |
| GATA6_human | 58 | CTGGAAAGGCTCTGGAGTCGCTGGCGTCCGTTTACATCATCATACATCATCAT |
| GATA6_human | 59 | AAGCGTAGGAACTGAGCAGCAGCGAGCGGGTTTACATCATCATACATCATCAT |
| GATA6_human | 60 | AGCAAGTCCTCCCAGCTCGACAGGTTGCCCTTTACATCATCATACATCATCAT |
| GATA6_human | 61 | GTCGCGGCTTGGTCGAGGTCAGTGAACAGCTTTACATCATCATACATCATCAT |
| GATA6_human | 62 | TACATCTCCTCCGGCTGCTCGGGTGCGAAGTTTACATCATCATACATCATCAT |
| GATA6_human | 63 | GTCTGGATGGAGCCGCAGTTCACGCACTCGTTTACATCATCATACATCATCAT |
| GATA6_human | 64 | TAGAGCCCGCAGGCGTTGCACAGGTAGTGGTTTACATCATCATACATCATCAT |
| GATA6_human | 65 | ATGAGGGGCCGGCTGAGGCCGTTCATCTTGTTTACATCATCATACATCATCAT |
| GATA6_human | 66 | TGGTGTGACAGTTGGCACAGGACAATCCAAGTTTACATCATCATACATCATCAT |
| GATA6_human | 67 | CGGCGTTTCTGCGCCATAAGGTGGTAGTTGTTTACATCATCATACATCATCAT |
| GATA6_human | 68 | GTCCACAAGCATTGCACACGGGTTCACCCTTTTACATCATCATACATCATCAT |
| GATA6_human | 69 | AGGTGGAAGTTGGAGTCATGGGAATGGAATTATTGCTTTTACATCATCATACATCATCAT |
| HMGA2_mouse | 70 | TAAAAGTGCAGCGTGAATGCAGAGATGGCAGTTTTACATCATCATACATCATCAT |
| HMGA2_mouse | 71 | CCGAACCAAGATAATGCACCTCGGCTAGCCTTTTACATCATCATACATCATCAT |
| HMGA2_mouse | 72 | GGTAGTATTGAGGAGTGGGGATTATTGCATCTCTGGCTTTACATCATCATACATCATCAT |
| HMGA2_mouse | 73 | TTTCTTCAACCACACACCCTTGCTTATCAGATCCACATTTACATCATCATACATCATCAT |
| HMGA2_mouse | 74 | TACTCAGCTAGTTATCTCCGATGCTTCAAGGCTGACATTTACATCATCATACATCATCAT |
| HMGA2_mouse | 75 | TTATGGCTAAGTGGCTTTTGGCTGTGTAGATTTCCCGTTTACATCATCATACATCATCAT |
| HMGA2_mouse | 76 | AGGCGGGAAAGAGAGCTTGTGGGCTTATCATTTACATCATCATACATCATCAT |
| HMGA2_mouse | 77 | AACAAGTGCAGGGCTGTAAATTCACTGTAGCATTCAGTTTACATCATCATACATCATCAT |
| HMGA2_mouse | 78 | TGGAGGTAGTTCCTTGCAAATCAGGAAGTCCATTTGTTTTACATCATCATACATCATCAT |
| HMGA2_mouse | 79 | CGTGGCAGTTAAGAATAACTGGTCACTGCAGTGTCTTTACATCATCATACATCATCAT |
| HMGA2_mouse | 80 | TATAGCAAAAGAGATTTCGGTTTCGCTCCTCCCATGTTTTACATCATCATACATCATCAT |
| HMGA2_mouse | 81 | ATCCAACTGATGCTGAGGTAGAAATTGAATGTCGGCGTTTACATCATCATACATCATCAT |
| HMGA2_mouse | 82 | CATCACTTGGGTGGGTTCATTGGGTACTGTTAACCTTTTACATCATCATACATCATCAT |
| HMGA2_mouse | 83 | CCCCTAATCCTCCTCTGCGGACTCTTGCGATTTACATCATCATACATCATCAT |
| HMGA2_mouse | 84 | AAGAGTACAGAGAAGAATGGTCGAGAACTGGGGATGGTTTACATCATCATACATCATCAT |
| HMGA2_mouse | 85 | TCAAAATGAAAGGTGTTACAGGATGTAACACGCAGGCTTTACATCATCATACATCATCAT |
| HMGA2_mouse | 86 | CTGAGATGCCGACCCAGTAAGGTTTGGTTTGGTTTACATCATCATACATCATCAT |
| HMGA2_mouse | 87 | TTCATCTGAGGTAAGTGGTACTGCGGTCTTTCACGTTTACATCATCATACATCATCAT |
| HMGA2_mouse | 88 | TTTTCCTTTCTCTTGCAGGGCTTGTGTCTAGCAGCTTTACATCATCATACATCATCAT |
| HMGA2_mouse | 89 | CTCCTGCTGCTGTGTATGAGTCCATAAATGGAGGATTTACATCATCATACATCATCAT |
| HMGA2_mouse | 90 | TGGTGTCCTAAGCAGATTTTCTATGATCATGGTGGGGTTTACATCATCATACATCATCAT |
| HMGA2_mouse | 91 | AATGTAACAATCTAGCAGGAAAGCTGCCACAAGCATTTTTACATCATCATACATCATCAT |
| HMGA2_mouse | 92 | GTGGCATCTGAGTGGACACAGAGTCACACACTTTACATCATCATACATCATCAT |
| HMGA2_mouse | 93 | CCCGGTCACCAGGGTGCCGGTTTAGGGTTTTTTACATCATCATACATCATCAT |
| HMGA2_mouse | 94 | ATAACTTCATCAGAGATGGAAATTGCTCGGTGTGCCGTTTACATCATCATACATCATCAT |
| HMGA2_mouse | 95 | GCATGTAGTGACTGATTAAGCCCACCACAGAGGTTTTACATCATCATACATCATCAT |
| HMGA2_mouse | 96 | AGTCTGGCCAACACGAGAAGCTGCTTCAGTTTTACATCATCATACATCATCAT |
| HMGA2_mouse | 97 | CGGGGCCCACAGAGGCTGTTATGTTTATTGTTTTACATCATCATACATCATCAT |
| HMGA2_mouse | 98 | TTTTATGAGCTGGCCAAAATGAGGTTTCTGTAAGGCCTTTACATCATCATACATCATCAT |
| HMGA2_mouse | 99 | ATAATCCACTAGAGAAGGTATTGCCACAAGCAAGCCGTTTACATCATCATACATCATCAT |
| HMGA2_mouse | 100 | TTTAATCTCTCCATAGCTCCTTTGGCGACTCACAACATTTACATCATCATACATCATCAT |
| HMGA2_mouse | 101 | GTGAGCCATCTGCCAGTCTCAGAGCGGCACTTTACATCATCATACATCATCAT |
| HMGA2_mouse | 102 | AACAGAGAACGAAGTCAGAGGGCACACAAAGGATTTACATCATCATACATCATCAT |
| HMGA2_mouse | 103 | GTGGCCTTTGAAGTAACCTCCCTGAAAACAGCTTTTACATCATCATACATCATCAT |
| HMGA2_mouse | 104 | ACGGGGAGAAAGTTGGATGCAGTTAATCGTCTTGATTTACATCATCATACATCATCAT |
| HMGA2_mouse | 105 | TACTGACACAAGCCACTATGAGAAACTACCTCCTGGCTTTACATCATCATACATCATCAT |
| HMGA2_mouse | 106 | TTTTGCTGCCTTTGGGTCTTCCTCTGGGTCTTTACATCATCATACATCATCAT |
| HMGA2_mouse | 107 | CCAGTGGGTAACACTCTGGAAAGTCAGCACACTTTACATCATCATACATCATCAT |
| HMGA2_mouse | 108 | CAGGTTGTCCCTGGGCTGATGTGGACGGCTTTTACATCATCATACATCATCAT |
| HMGA2_mouse | 109 | ACACAAGAAGCGTCCAAAACAAATCTGCTTGAGACATTTACATCATCATACATCATCAT |
| HMGA2_mouse | 110 | TTTTCATTGCATTAGGCAAAAGGCTGAGCTGGTTGTTTTACATCATCATACATCATCAT |
| HMGA2_mouse | 111 | CGCCCAGCACCTTTCGGGAGACGGGATGTATTTACATCATCATACATCATCAT |
| HMGA2_mouse | 112 | AAATGTTTCAATGAGGGAGAGGTGAGGTTTGAGCTCCTTTACATCATCATACATCATCAT |
| HMGA2_mouse | 113 | GAGAGCTGGAGAGGGCAAGAGCGGCGAGAGTTTACATCATCATACATCATCAT |
| HMGA2_mouse | 114 | TGAGTGATATAGGCTGCAAGTTCGTTCCTTTGGAAGTTTTACATCATCATACATCATCAT |
| HMGA2_mouse | 115 | GAGTGCGCCGGGGACTACTGCTGCTGCTTATTTACATCATCATACATCATCAT |
| GATA6_mouse | 116 | TAAACAAACAGCGCTAGCCGAAGGGTGCCAGGCTTTACATCATCATACATCATCAT |
| GATA6_mouse | 117 | CGGGGTCTGCTAGTCGCTGCTGGTGAATAAAATTTACATCATCATACATCATCAT |
| GATA6_mouse | 118 | GTCCGGCGCTACTCCAACCTGACTTTTGATTTTTACATCATCATACATCATCAT |
| GATA6_mouse | 119 | CCTGCGCTCCTGATTGGACTCACCGAGCCCTTTACATCATCATACATCATCAT |
| GATA6_mouse | 120 | GCCTCTTACTGCTCTGCCGGAAAACTGCAGTTTACATCATCATACATCATCAT |
| GATA6_mouse | 121 | TCCTGCAAAAGCCCATCTCTTCTTACTTCAGTAAGCTTTTACATCATCATACATCATCAT |
| GATA6_mouse | 122 | GAGTCAGAGGCAGGAAGCAGCCCAGGCTGGTTTACATCATCATACATCATCAT |
| GATA6_mouse | 123 | TGTCACAGTTTCTCCCACTGGTATGGGGCATTTACATCATCATACATCATCAT |
| GATA6_mouse | 124 | ATCTTCCTTAGCAGACAAGGCCCCGGTCATTTTACATCATCATACATCATCAT |
| GATA6_mouse | 125 | CGAGGTTGATCACAAGAAGCACATGATTTGGAGCAATTTTACATCATCATACATCATCAT |
| GATA6_mouse | 126 | GGTGGTCGGGGATGAATGGGTTCTGGGATAACTTTACATCATCATACATCATCAT |
| GATA6_mouse | 127 | TCCTCTCCACGAACGCTTGTGAAATGTGCACTTTACATCATCATACATCATCAT |
| GATA6_mouse | 128 | CCTCCAGGATAGACCAAATGGCTCCCAGTGCTTTACATCATCATACATCATCAT |
| GATA6_mouse | 129 | GCCACGTTACGATGAACGTTGAGATAAGTCAGTCAGTTTTACATCATCATACATCATCAT |
| GATA6_mouse | 130 | CACAGAAGTGGGCTGTGAGTGTAAGAAGCATATGTCTTTTACATCATCATACATCATCAT |
| GATA6_mouse | 131 | TCAGTAAAAGAACGGGGACTGTGTTGGTGTTCTTGTATTTACATCATCATACATCATCAT |
| GATA6_mouse | 132 | CACGTGGTACAGGCGTCAAGAGTGTTACAGATACTTTTTACATCATCATACATCATCAT |
| GATA6_mouse | 133 | GAGCAGGAGGAGGACGAAGACGAGATGGGGTTTACATCATCATACATCATCAT |
| GATA6_mouse | 134 | GGGATGCGAGGCGTAGGGGCTGAGCAAGAGTTTACATCATCATACATCATCAT |
| GATA6_mouse | 135 | AACAGGTCCTCCCAAGTCGACAGGGCGCTCTTTACATCATCATACATCATCAT |
| GATA6_mouse | 136 | GTCGCGGCCTGATCGAGGTCAGTGAAGAGCTTTACATCATCATACATCATCAT |
| GATA6_mouse | 137 | TACATTTCCTCCGGCTGCTCGGCCGCGAAGTTTACATCATCATACATCATCAT |
| GATA6_mouse | 138 | TGAAGGTAGGGCAGGCCGGACAGCATGGAGTTTACATCATCATACATCATCAT |
| GATA6_mouse | 139 | GAGCTGTACTGGTGCTCCCGGCCACCCATGTTTACATCATCATACATCATCAT |
| GATA6_mouse | 140 | ATGGTGGTGGTGGTACGTTCCGTTCAGCGGCTTTACATCATCATACATCATCAT |
| GATA6_mouse | 141 | CATGTAGGGCGAGTAGGTCGGGTGATGGTGTTTACATCATCATACATCATCAT |
| GATA6_mouse | 142 | GAAGGGTCCTGCTGGCCAGGCAGGAGTCAGTTTACATCATCATACATCATCAT |
| GATA6_mouse | 143 | GCCCTGTAAGCTGTGGAGCACCGGCGTTTCTTTACATCATCATACATCATCAT |
| GATA6_mouse | 144 | GTCTGGATGGAGCCGCAGTTCACGCACTCGTTTACATCATCATACATCATCAT |
| GATA6_mouse | 145 | ATGACCGGTGCCGTCTCGTCTCCACAGTGGTTTACATCATCATACATCATCAT |
| GATA6_mouse | 146 | GCTGTAGAGACCGCATGCATTGCACAGGTATTTACATCATCATACATCATCAT |
| GATA6_mouse | 147 | GATGAGGGGCCTGCTGAGGCCATTCATCTTTTTACATCATCATACATCATCAT |
| GATA6_mouse | 148 | AGTGGTTGTGGTGTGACAGTTGGCACAGGATTTACATCATCATACATCATCAT |
| GATA6_mouse | 149 | GGCTCACCCTCAGCATTTCTACGCCATAAGGTTTTACATCATCATACATCATCAT |
| GATA6_mouse | 150 | AGTTTCATATAGAGCCCGCAAGCATTGCACACATTTACATCATCATACATCATCAT |
| GATA6_mouse | 151 | GTAGGAGTCATAGGGACAGAGCCACTGCTGTTTTACATCATCATACATCATCAT |
| GATA6_mouse | 152 | CCTGAGGTGGTCGCTTGTGTAGAAGGAGAAGTATTTTTTTACATCATCATACATCATCAT |
| GATA6_mouse | 153 | GGCGTTTTCTCCCACTGCAGACATCACTGATGTTTACATCATCATACATCATCAT |
| GATA6_mouse | 154 | ACCTGAATACTTGAGGTCACTGTTCTCGGGGTTTTTACATCATCATACATCATCAT |
| GATA6_mouse | 155 | GGACAGACTGACACCTATGTAGAGGCCGTCTTGTTTACATCATCATACATCATCAT |
| GATA6_mouse | 156 | GTCGCACGGAGGATGTGACTTCGGCAGGGGTTTACATCATCATACATCATCAT |
| GATA6_mouse | 157 | AGGCCAGGGCCAGAGCACACCAAGAATCCTTTTACATCATCATACATCATCAT |
| GATA6_mouse | 158 | GCCCTCCTTGCCTCTTGGTAGCACCAGCTCTTTACATCATCATACATCATCAT |
| GATA6_mouse | 159 | TGGACAATATCAGACACAAGTGGTATGAGGCCTTCAGTTTACATCATCATACATCATCAT |
| GATA6_mouse | 160 | GCACAGAAATCACGCATCGAAGGAATGTTATGTCTGCTTTACATCATCATACATCATCAT |
| p27 | 161 | ACATCATCATGGGCCTTTTGGCCCATGATGATGTATGATGATG TTTTTTT |
| CleanG | 162 | CCCCGAAAGTGGCCTCGGGCCTTTTGGCCCGAGGCCACTTTCG |
| p27 fluor | 163 | ttATGATGATGTATGATGATGT |
| (Alexa 488) | | with a 5′ ATTO488N fluorophore and a 3′ inverted dT |
| ITS | 164 | TCGAGACGCCCTAGCGGGAA |
| ITS | 165 | GAACGCGCTAGGTACCTGGA |
| ITS | 166 | CAAGGGGTCTTTAAACCTCC |
| ITS | 167 | GTCGGAAGGTTTCACACCAC |
| ITS | 168 | CAGCAAACGGGACCGGACTC |

While illustrative embodiments have been illustrated and described, it will be appreciated that various changes can be made therein without departing from the spirit and scope of the invention.

1. A method of treating a subject identified as suffering from Pancreatic Ductal Adenocarcinoma (PDA), the method comprising:
administering to the subject an effective amount of at least one therapeutic compound as a first-line treatment, wherein the subject is identified as suffering from PDA by a method comprising:
(i) obtaining one or more biological sample from the subject;
(ii) determining/quantifying/measuring an expression level of at least one biomarker in the one or more biological sample obtained from the subject; and
(iii) comparing the expression level of the at least one biomarker in the one or more biological sample obtained from the subject with an expression level of the at least one biomarker in a reference/control sample,
wherein the at least one biomarker comprises High mobility Group A2 (HMGA2), and wherein a differential expression of the at least one biomarker in the biological sample of the subject relative to the expression of the biomarker in the reference/control sample identifies the subject as suffering from PDA.

2. The method of claim 1, wherein the reference sample is a biological sample obtained from a healthy subject not suffering from PDA.

3. The method of claim 1, wherein the reference sample is a biological sample corresponding to the biological sample obtained from the subject suffering from PDA.

4. The method of claim 1, wherein the one or more biological sample comprises a biopsy tissue or resected tissue of the Pancreatic Ductal Adenocarcinoma.

5. The method of claim 1, wherein the differential expression comprises an overexpression of HMGA2 (HMGA2^{high}) in the one or more biological sample relative to the reference/control sample, and wherein the at least one therapeutic compound comprises FOLFIRINOX.

6. The method of claim 1, wherein the differential expression comprises a lower expression of HMGA2 (HMGA2^{low}) in the one or more biological sample relative to the reference/control sample, and wherein the at least one therapeutic compound comprises Gemcitabine (GA).

7. The method of claim 1, further comprising determining/quantifying/measuring an expression level of at least one other biomarker in the biological sample obtained from the subject and comparing the expression level of the at least one other biomarker in the one or more biological sample obtained from the subject with an expression level of the at least one other biomarker in a reference/control sample.

8. The method of claim 7, wherein the at least one other biomarker comprises KRT17, KRT5, S100A2, GATA binding protein 6 (GATA6), ECAD, CLDN18.2 and TTF1, kirsten rat sarcoma viral oncogene homolog (KRAS), or a combination thereof.

9. The method of claim 7, wherein the at least one other biomarker comprises GATA binding protein 6 (GATA6).

10. The method of claim 9, wherein the differential expression comprises an overexpression of HMGA2 (HMGA2^{high}) and a lower expression of GATA6 (GATA6^{low}) relative to the expression of HMGA2 and GATA6, respectively, in the reference/control sample, and wherein the at least one therapeutic compound comprises FOLFIRINOX.

11. The method of claim 9, wherein the differential expression comprises a lower expression of HMGA2 (HMGA2^{low}) and an overexpression of GATA6 (GATA6^{high}) relative to the expression of HMGA2 and GATA6, respectively, in the reference/control sample, and wherein the at least one therapeutic compound comprises Gemcitabine.

12-26. (canceled)

27. A method of increasing overall median survival in a subject suffering from Pancreatic Ductal Adenocarcinoma (PDA), the method comprising:
administering to the subject an effective amount of at least one PDA subtype specific therapeutic compound as a first-line treatment, wherein the subject has been diagnosed with a PDA subtype by a method comprising the steps of:
(i) obtaining one or more biological sample from the subject;
(ii) determining/quantifying/measuring an expression level of at least one biomarker in the one or more biological sample obtained from the subject; and
(iii) comparing the expression level of the at least one biomarker in the one or more biological sample obtained from the subject with an expression level of the at least one biomarker in a reference/control sample,
wherein the at least one biomarker comprises High mobility Group A2 (HMGA2), and wherein a differential expression of the at least one biomarker in the biological sample of the subject relative to the expression of the biomarker in the reference/control sample identifies the subject as suffering from the subtype of PDA.

28. The method of claim 27, wherein the differential expression comprises an overexpression of HMGA2 (HMGA2^{high}) in the one or more biological sample relative to the reference/control sample, and wherein the at least one PDA subtype specific therapeutic compound administered as a first-line treatment comprises FOLFIRINOX.

29. The method of claim 28, wherein the subject is diagnosed with basal subtype PDA.

30. The method of claim 27, wherein the differential expression comprises a lower expression of HMGA2 (HMGA2^{low}) in the one or more biological sample relative to the reference/control sample, and wherein the at least one PDA subtype specific therapeutic compound administered as a first-line treatment comprises Gemcitabine.

31. The method of claim 30, wherein the subject is diagnosed with classical subtype PDA.

32. The method of claim 27, further comprising determining/quantifying/measuring the expression of at least one other biomarker in the biological sample obtained from the subject and comparing the expression levels of the at least one other biomarker in the one or more biological sample obtained from the subject with an expression level of the at least one other biomarker in a reference/control sample, wherein the at least one other biomarker comprises KRT17, KRT5, S100A2, GATA binding protein 6 (GATA6), ECAD, CLDN18.2 and TTF1, kirsten rat sarcoma viral oncogene homolog (KRAS), or a combination thereof.

33. (canceled)

34. The method of claim 32, wherein the at least one other biomarker comprises GATA binding protein 6 (GATA6).

35. The method of claim 34, wherein the differential expression comprises an overexpression of HMGA2 (HMGA2^{high}) and a lower expression of GATA6 (GATA6^{low}) relative to the expression of HMGA2 and GATA6, respectively, in a reference/control sample, wherein the subject is diagnosed with basal subtype PDA, and wherein the at least one PDA subtype specific therapeutic compound administered as a first-line treatment comprises FOLFIRINOX.

36. The method of claim 34, wherein the differential expression comprises a lower expression of HMGA2 (HMGA2^{low}) and an overexpression of GATA6 (GATA6^{high}) relative to the expression of HMGA2 and GATA6, respectively, in a reference/control sample, wherein the subject is diagnosed with classical subtype PDA, and wherein the at least one therapeutic compound administered as a first-line treatment comprises Gemcitabine.
